(19) United States
(12) Patent Application Publication
(10) Pub. No.: US 2016/0190479 A1
(43) Pub. Date: Jun. 30, 2016

(54) Light-Emitting Element, Organic Compound, Light-Emitting Device, Electronic Device, and Lighting Device

(71) Applicant: Semiconductor Energy Laboratory Co., Ltd., Kanagawa-ken (JP)

(72) Inventors: Tomoka HARA, Ebina (JP); Hideko INOUE, Atsugi (JP); Tatsuyoshi TAKAHASHI, Atsugi (JP); Takao HAMADA, Atsugi (JP); Satoshi SEO, Sagamihara (JP)

(73) Assignee: Semiconductor Energy Laboratory Co., Ltd., Kanagawa-ken (JP)

(21) Appl. No.: 14/976,244

(22) Filed: Dec. 21, 2015

(30) Foreign Application Priority Data
Dec. 26, 2014 (JP) 2014-264831

(51) Int. Cl. H01L 51/00; C07D 403/14; C07D 409/10; C09K 11/02; C09K 11/06

(52) U.S. Cl. CPC H01L 51/0072; H01L 51/0074; H01L 51/006; H01L 51/0085; C09K 11/025; C09K 11/06; C07D 409/10; C07D 403/14; H01L 51/5016

(57) ABSTRACT

The organic compound represented by Formula G1 below has a structure in which the 2-position of a dibenzoquinazoline ring is directly, or via one or more arylene groups, bonded to a skeleton with a hole-transport property.

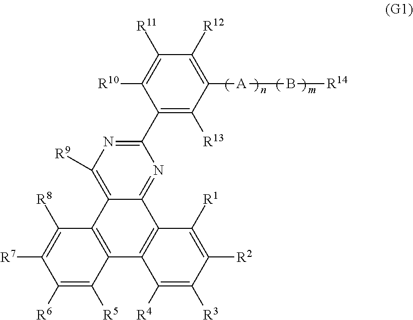

In the formula, n represents any of 0 to 3, m represents 1 or 2, A represents a single bond, or a substituted or unsubstituted arylene group having 6 to 13 carbon atoms, B represents a ring having a substituted or unsubstituted dibenzofuran skeleton, a substituted or unsubstituted dibenzothiophene skeleton, or a substituted or unsubstituted carbazole skeleton, and each of R¹ to R¹⁴ independently represents any of hydrogen, a substituted or unsubstituted alkyl group having 1 to 6 carbon atoms, a substituted or unsubstituted cycloalkyl group having 5 to 7 carbon atoms, and a substituted or unsubstituted aryl group having 6 to 13 carbon atoms.

### BACKGROUND OF THE INVENTION

1. Field of the Invention

One embodiment of the present invention relates to an object, a substance, a method, or a manufacturing method. In addition, one embodiment of the present invention relates to a process, a machine, manufacture, or a composition of matter. In particular, one embodiment of the present invention relates to a light-emitting element, an organic compound, a light-emitting device, an electronic device, and a lighting device. Note that one embodiment of the present invention is not limited to the above technical fields. More specific examples of the technical field of one embodiment of the present invention disclosed in this specification include a semiconductor device, a display device, a liquid crystal display device, a power storage device, a memory device, an imaging device, a method for driving any of them, and a method for manufacturing any of them.

2. Description of the Related Art

The use of light-emitting elements, which include organic compounds as luminous bodies and are characterized by their thinness, lightweight, high speed response, low driving voltage, etc., in the next-generation flat panel displays is expected. In particular, a light-emitting device in which a plurality of light-emitting elements are arranged is considered to have advantages in a wide viewing angle and excellent visibility over a conventional liquid crystal display device.

It is said that the light emission mechanism of a light-emitting element is as follows: when a voltage is applied between a pair of electrodes with an EL layer including a luminous body provided therebetween, electrons injected from the cathode and holes injected from the anode recombine in the light emission center of the EL layer to form molecular excitons, and energy is released and light is emitted when the molecular excitons relax to the ground state. Singlet excitation and triplet excitation are known as excited states, and light emission is considered achievable through either of the excited states.

In order to improve element characteristics of such light-emitting elements, improvement of an element structure, development of a material, and the like have been actively carried out (see Patent Document 1, for example).

### REFERENCE

### Patent Document

[Patent Document 1] Japanese Published Patent Application No. 2010-182699

### SUMMARY OF THE INVENTION

In development of a light-emitting element, an organic compound used in the light-emitting element is very important for improving the characteristics and reliability. Thus, an object of one embodiment of the present invention is to provide a novel organic compound. That is, a novel organic compound that is effective in improving the element characteristics and reliability is provided. Another object is to provide a novel organic compound that can be used in a light-emitting element. Another object is to provide a novel organic compound that can be used in an EL layer of a light-emitting element. Another object is to provide a highly efficient, reliable, and novel light-emitting element using a novel organic compound of one embodiment of the present invention. Another object is to provide a novel light-emitting device, a novel electronic device, or a novel lighting device. Note that the descriptions of these objects do not preclude the existence of other objects. In one embodiment of the present invention, there is no need to achieve all the objects. Other objects will be apparent from and can be derived from the description of the specification, the drawings, the claims, and the like.

One embodiment of the present invention is a light-emitting element including an anode, a cathode, and an EL layer between the anode and the cathode, in which the EL layer includes a light-emitting layer, the light-emitting layer includes a first organic compound and a light-emitting substance, and the first organic compound includes a dibenzoquinazoline ring and a skeleton with a hole-transport property.

Another embodiment of the present invention is a light-emitting element including an anode, a cathode, and an EL layer between the anode and the cathode, in which the EL layer includes a light-emitting layer, the light-emitting layer includes a first organic compound, a second organic compound with a hole-transport property, and a light-emitting substance, and the first organic compound includes a dibenzoquinazoline ring and a skeleton with a hole-transport property.

Another embodiment of the present invention is a light-emitting element including an anode, a cathode, and an EL layer between the anode and the cathode, in which the EL layer includes a light-emitting layer, the light-emitting layer includes a first organic compound and a light-emitting substance, and the first organic compound has a structure in which the 2-position of a dibenzoquinazoline ring is directly bonded to a skeleton with a hole-transport property or a structure in which the 2-position of a dibenzoquinazoline ring is bonded to a skeleton with a hole-transport property via one or more arylene groups.

Another embodiment of the present invention is a light-emitting element including an anode, a cathode, and an EL layer between the anode and the cathode, in which the EL layer includes a light-emitting layer, the light-emitting layer includes a first organic compound, a second organic compound with a hole-transport property, and a light-emitting substance, and the first organic compound has a structure in which the 2-position of a dibenzoquinazoline ring is directly bonded to a skeleton with a hole-transport property or a structure in which the 2-position of a dibenzoquinazoline ring is bonded to a skeleton with a hole-transport property via one or more arylene group.

In each of the above structures, the skeleton with a hole-transport property is a diarylamino group or a π-electron rich heteroaromatic ring. In addition, the π-electron rich heteroaromatic ring includes a five-membered heteroaromatic ring.

In each of the above structures, the skeleton with a hole-transport property is a ring having a dibenzofuran skeleton, a dibenzothiophene skeleton, or a carbazole skeleton.

In each of the above structures, the skeleton with a hole-transport property has a structure in which a plurality of rings selected from rings each having a dibenzofuran skeleton, a dibenzothiophene skeleton, or a carbazole skeleton are bonded to each other.

In each of the above structures, the light-emitting substance is a phosphorescent compound.

In each of the above structures, the first organic compound is represented by General Formula (G1).

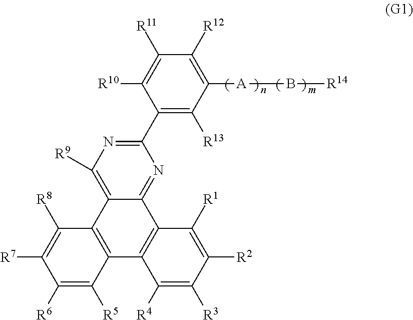

In General Formula (G1), n represents any of 0 to 3, and m represents 1 or 2. Further, A represents a single bond, or a substituted or unsubstituted arylene group having 6 to 13 carbon atoms, B represents a substituted or unsubstituted dibenzofuran skeleton, a substituted or unsubstituted dibenzothiophene skeleton, or a substituted or unsubstituted carbazole skeleton, and each of R¹ to R¹⁴ independently represents any of hydrogen, a substituted or unsubstituted alkyl group having 1 to 6 carbon atoms, a substituted or unsubstituted cycloalkyl group having 5 to 7 carbon atoms, and a substituted or unsubstituted aryl group having 6 to 13 carbon atoms.

The first organic compound represented by General Formula (G1) has a structure in which the 2-position of a dibenzoquinazoline ring is bonded to a skeleton with a hole-transport property via a 1,3-phenylene group, which is preferable because the singlet level (S1) and the triplet level (T1) can be raised and an energy gap can be widened. Furthermore, as compared with an organic compound having a structure in which the 2-position of a dibenzoquinazoline ring is bonded to a skeleton with a hole-transport property via a 1,4-phenylene group, the first organic compound represented by General Formula (G1) is preferable in obtaining a light-emitting element with high emission efficiency because its use in the light-emitting element makes it easy to adjust the carrier balance and prevents carrier passage.

Another embodiment of the present invention is an organic compound including a dibenzoquinazoline ring and a skeleton with a hole-transport property, more preferably, an organic compound having a structure in which the 2-position of a dibenzoquinazoline ring is directly bonded to a skeleton with a hole-transport property or a structure in which the 2-position of a dibenzoquinazoline ring is bonded to a skeleton with a hole-transport property via one or more arylene groups.

Another embodiment of the present invention is an organic compound represented by General Formula (G1).

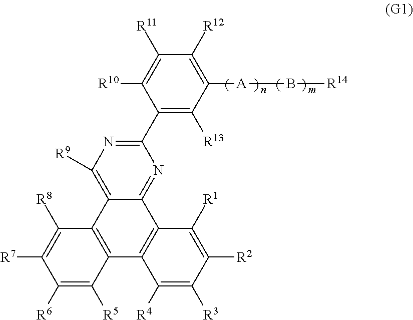

In General Formula (G1), n represents any of 0 to 3, and m represents 1 or 2. Further, A represents a single bond, or a substituted or unsubstituted arylene group having 6 to 13 carbon atoms, B represents a ring having a substituted or unsubstituted dibenzofuran skeleton, a ring having a substituted or unsubstituted dibenzothiophene skeleton, or a ring having a substituted or unsubstituted carbazole skeleton, and each of R¹ to R¹⁴ independently represents any of hydrogen, a substituted or unsubstituted alkyl group having 1 to 6 carbon atoms, a substituted or unsubstituted cycloalkyl group having 5 to 7 carbon atoms, and a substituted or unsubstituted aryl group having 6 to 13 carbon atoms.

Another embodiment of the present invention is the organic compound represented by General Formula (G1) in which B is represented by any one of General Formulae (B1) to (B4).

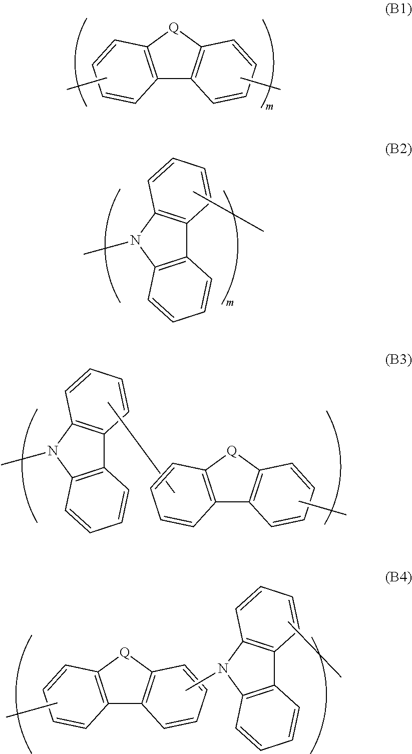

In General Formulae (B1) to (B4), m represents 1 or 2. Further, Q represents any of S, N—R¹⁵, and O, and R¹⁵ represents hydrogen, or a substituted or unsubstituted phenyl group. A benzene ring in (B1) to (B4) may have a substituent, and the substituent is any of a substituted or unsubstituted alkyl group having 1 to 6 carbon atoms, a substituted or unsubstituted cycloalkyl group having 5 to 7 carbon atoms, and a substituted or unsubstituted aryl group having 6 to 13 carbon atoms.

Another embodiment of the present invention is an organic compound represented by Structural Formula (100) or Structural Formula (123).

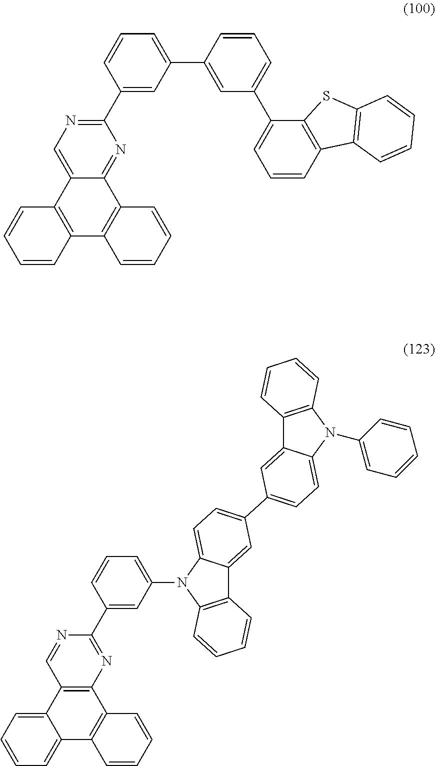 [SMILES: c1cc(-c2cccc(-c3cccc4c3sc3ccccc34)c2)cc(-c2ncc3c4ccccc4c4ccccc4c3n2)c1 ; c1ccc(-n2c3ccccc3c3cc(-c4ccc5c(c4)c4ccccc4n5-c4cccc(-c5ncc6c7ccccc7c7ccccc7c6n5)c4)ccc32)cc1]

The organic compound of one embodiment of the present invention can be used in combination with a light-emitting substance that emits phosphorescence (phosphorescent compound) in a light-emitting layer of a light-emitting element, which is very effective because light emission from a triplet excited state can be obtained from the light-emitting layer and thus the efficiency of the light-emitting element can be improved. Accordingly, embodiments of the present invention include a light-emitting element in which the organic compound of one embodiment of the present invention and a phosphorescent compound are used in a light-emitting layer. Furthermore, the light-emitting layer may additionally contain a third substance.

In addition, the present invention includes, in its scope, a light-emitting device with the light-emitting element, and also a lighting device with the light-emitting device. The light-emitting device in this specification refers to an image display device and a light source (e.g., a lighting device). In addition, the light-emitting device includes, in its category, all of a module in which a light-emitting device is connected to a connector such as a flexible printed circuit (FPC), a tape carrier package (TCP), a module in which a printed wiring board is provided on the tip of a TCP, and a module in which an integrated circuit (IC) is directly mounted on a light-emitting element by a chip on glass (COG) method.

According to one embodiment of the present invention, a novel organic compound can be provided. In other words, a novel organic compound that is effective in improving the element characteristics and reliability can be provided. In addition, a novel organic compound that can be used in a light-emitting element can be provided. In addition, a novel organic compound that can be used in an EL layer of a light-emitting element can be provided. In addition, a highly efficient, reliable, and novel light-emitting element using a novel organic compound of one embodiment of the present invention can be provided. In addition, a novel light-emitting device, a novel electronic device, or a novel lighting device can be provided. Note that the description of these effects does not preclude the existence of other effects. One embodiment of the present invention does not necessarily achieve all the effects listed above. Other effects will be apparent from and can be derived from the description of the specification, the drawings, the claims, and the like.

### BRIEF DESCRIPTION OF THE DRAWINGS

In the accompanying drawings:

FIGS. 1A and 1B illustrate structures of light-emitting elements;

FIGS. 2A and 2B illustrate structures of light-emitting elements;

FIGS. 3A to 3C illustrate light-emitting devices;

FIGS. 4A to 4D, 4D′-1, and 4D′-2 illustrate electronic devices;

FIGS. 5A to 5C illustrate an electronic device;

FIGS. 6A to 6D illustrate lighting devices;

FIG. 7 illustrates lighting devices;

FIGS. 8A and 8B illustrate an example of a touch panel;

FIGS. 9A and 9B illustrate an example of a touch panel;

FIGS. 10A and 10B illustrate an example of a touch panel;

FIGS. 11A and 11B are a block diagram and a timing chart of a touch sensor;

FIG. 12 is a circuit diagram of a touch sensor;

FIG. 13 is ¹H-NMR chart of the organic compound represented by Structural Formula (100);

FIG. 14 shows an ultraviolet-visible absorption spectrum and an emission spectrum of the organic compound represented by Structural Formula (100) in a toluene solution;

FIG. 15 shows an ultraviolet-visible absorption spectrum and an emission spectrum of a solid thin film of the organic compound represented by Structural Formula (100);

FIG. 16 illustrates a light-emitting element;

FIG. 17 shows current density-luminance characteristics of Light-emitting Element 1;

FIG. 18 shows voltage-luminance characteristics of Light-emitting Element 1;

FIG. 19 shows luminance-current efficiency characteristics of Light-emitting Element 1;

FIG. 20 shows voltage-current characteristics of Light-emitting Element 1;

FIG. 21 shows an emission spectrum of Light-emitting Element 1;

FIG. 22 shows ¹H-NMR chart of the organic compound represented by Structural Formula (123);

FIG. 23 shows ultraviolet-visible absorption spectrum and emission spectrum of the organic compound represented by the structural formula (123) in a toluene solution;

FIG. 24 shows ultraviolet-visible absorption spectrum and emission spectrum of a solid thin film of the organic compound represented by Structural Formula (123);

FIG. 25 shows current density-luminance characteristics of Light-emitting Element 2;

FIG. 26 shows voltage-luminance characteristics of Light-emitting Element 2;

FIG. 27 shows luminance-current efficiency characteristics of Light-emitting Element 2;

FIG. 28 shows voltage-current characteristics of Light-emitting Element 2; and

FIG. 29 shows an emission spectrum of Light-emitting Element 2.

### DETAILED DESCRIPTION OF THE INVENTION

Embodiments of the present invention will be described in detail below with reference to the drawings. However, the present invention is not limited to the description below, and it is to be easily understood that modes and details thereof can be variously modified without departing from the purpose and the scope of the present invention. Accordingly, the present invention should not be interpreted as being limited to the content of the embodiments below.

Note that the terms “film” and “layer” can be interchanged with each other depending on the case or circumstances. For example, the term “conductive layer” can be changed into the term “conductive film” in some cases, and the term “insulating film” can be changed into the term “insulating layer” in some cases.

### Embodiment 1

In this embodiment, organic compounds of embodiments of the present invention are described.

An organic compound of one embodiment of the present invention has a dibenzoquinazoline ring and a skeleton with a hole-transport property. Alternatively, the organic compound of one embodiment of the present invention has a dibenzoquinazoline ring and a skeleton with a hole-transport property, and the 2-position of the dibenzoquinazoline ring is directly bonded to the skeleton with the hole-transport property or is bonded to the skeleton via one or more arylene groups. Since a dibenzoquinazoline ring has an electron-transport property, the organic compound of one embodiment of the present invention is a compound having a hole-transport property and an electron-transport property. Therefore, the organic compound of one embodiment of the present invention is excellent in carrier-transport property and useful as an organic compound used for a light-emitting element or the like.

The skeleton with a hole-transport property is preferably a diarylamino group or a π-electron rich heteroaromatic ring. A π-electron rich heteroaromatic ring refers to a ring in which an unshared electron pair of heteroatoms increases the π-electron density, and preferably contains a five-membered heteroaromatic ring having one heteroatom, such as furan, thiophene, and pyrrole.

More specifically, a ring having a dibenzofuran skeleton, a dibenzothiophene skeleton, or a carbazole skeleton is preferable as the skeleton with a hole-transport property because of their excellence in heat resistance and chemical stability. In particular, when the skeleton with a hole-transport property contains a plurality of rings which are independently selected from a ring having a dibenzofuran skeleton, a ring having a dibenzothiophene skeleton, and a ring having a carbazole skeleton, and then the plurality of rings are bonded to one another, the hole-transport property as well as the heat resistance increases. Note that rings having a dibenzofuran skeleton, a dibenzothiophene skeleton, or a carbazole skeleton according to one embodiment of the present invention include, in its category, a dibenzofuran ring, a dibenzothiophene ring, and a carbazole ring each condensed with a benzene ring, a naphthalene ring, or the like. That is, the skeleton with a hole-transport property includes, in its category, a dibenzofuran ring, a dibenzothiophene ring, a carbazole ring, a benzonaphthofuran ring, a dinaphthofuran ring, a benzonaphthothiophene ring, a dinaphthothiophene ring, a benzocarbazole ring, a dibenzocarbazole ring, and the like.

One embodiment of the organic compound described in this embodiment is an organic compound having a structure represented by General Formula (G1).

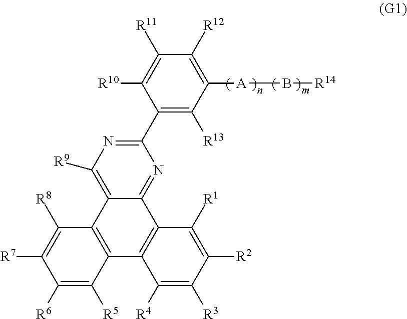

In General Formula (G1), n represents any of 0 to 3, and m represents 1 or 2. Further, A represents a single bond, or a substituted or unsubstituted arylene group having 6 to 13 carbon atoms; B represents a ring having a substituted or unsubstituted dibenzofuran skeleton, a ring having a substituted or unsubstituted dibenzothiophene skeleton, or a ring having a substituted or unsubstituted carbazole skeleton; and each of R¹ to R¹⁴ independently represents any of hydrogen, a substituted or unsubstituted alkyl group having 1 to 6 carbon atoms, a substituted or unsubstituted cycloalkyl group having 5 to 7 carbon atoms, and a substituted or unsubstituted aryl group having 6 to 13 carbon atoms. More specifically, B represents a substituted or unsubstituted dibenzofuran ring, a substituted or unsubstituted dibenzothiophene ring, or a substituted or unsubstituted carbazole ring. The term “substitute” in the above structures preferably refers to substitution by a substituent such as an alkyl group having 1 to 6 carbon atoms, e.g., a methyl group, an ethyl group, an n-propyl group, an iso-propyl group, a sec-butyl group, a tert-butyl group, an n-pentyl group, or an n-hexyl group, or substitution by a substituent such as an aryl group having 6 to 12 carbon atoms, e.g., a phenyl group, an o-tolyl group, an m-tolyl group, a p-tolyl group, a 1-napthly group, a 2-naphthyl group, a 2-biphenyl group, a 3-biphenyl group, or a 4-biphenyl group. These substituents may be bonded to each other to form a ring. For example, in the case where the arylene group is a 2,7-fluorenylene group having two phenyl groups at the 9-position as a substituent, the phenyl groups may be bonded to each other to become a spiro-9,9′-bifluorene-2,7-diyl group.

Examples of the substituted or unsubstituted arylene group having 6 to 13 carbon atoms in General Formula (G1) above include a substituted or unsubstituted phenylene group, a substituted or unsubstituted biphenylene group, a substituted or unsubstituted naphthylene group, a substituted or unsubstituted fluorenylene group, and a substituted or unsubstituted phenanthrenylene group. Specific examples of the substituent in the case where these groups further include a substituent are as described above. More specifically, a 1,2-phenylene group, a 1,3-phenylene group, a 1,4-phenylene group, a 2,6-toluylene group, a 3,5-toluylene group, a 2,4-toluylene group, a 4,6-dimethylbenzene-1,3-diyl group, a 2,4,6-trimethylbenzene-1,3-diyl group, a 2,3,5,6-tetramethylbenzene-1,4-diyl group, a 3,3′-biphenylene group, a 3,4′-biphenylene group, a 4,4′-biphenylene group, a 1,4-naphthylene group, a 1,5-naphthylene group, a 2,6-naphthylene group, a 2,7-naphthylene group, a 2,7-fluorenylene group, a 9,9-dimethyl-2,7-fluorenylene group, a 9,9-diphenyl-2,7-fluorenylene group, a 9,9-dimethyl-1,4-fluorenylene group, a spiro-9,9′-bifluorene-2,7-diyl group, a 9,10-dihydro-2,7-phenanthrylene group, and the like can be given.

In General Formula (G1), B is preferably represented by any one of General Formulae (B 1) to (B4) below.

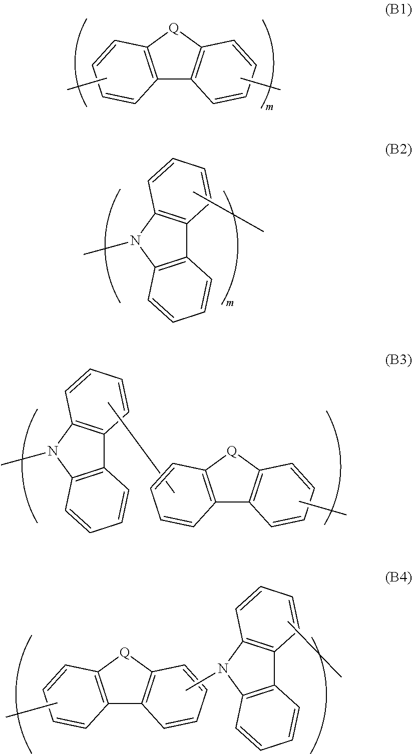

In the formulae, in represents 1 or 2, and Q represents any of S, N—R¹⁵, and O. Further, R¹⁵ represents hydrogen, a substituted or unsubstituted phenyl group. The benzene ring in (B1) to (B4) may have a substituent, and the substituent is any of an alkyl group having 1 to 6 carbon atoms, a cycloalkyl group having 5 to 7 carbon atoms, and a substituted or unsubstituted aryl group having 6 to 13 carbon atoms.

The term “substitute” in the above structures preferably refers to substitution by a substituent such as an alkyl group having 1 to 6 carbon atoms, e.g., a methyl group, an ethyl group, an n-propyl group, an iso-propyl group, a sec-butyl group, a tert-butyl group, an n-pentyl group, or an n-hexyl group, or substitution by a substituent such as an aryl group having 6 to 12 carbon atoms, e.g., a phenyl group, an o-tolyl group, an m-tolyl group, a p-tolyl group, a 1-napthly group, a 2-naphthyl group, a 2-biphenyl group, a 3-biphenyl group, or a 4-biphenyl group. These substituents may be bonded to each other to form a ring. For example, in the case where the aryl group is a 2-fluorenyl group having two phenyl groups at the 9-position as a substituent, the phenyl groups may be bonded to each other to become a Spiro-9,9′-bifluoren-2-yl group.

Another embodiment of the organic compound described in this embodiment is an organic compound represented by General Formula (G2).

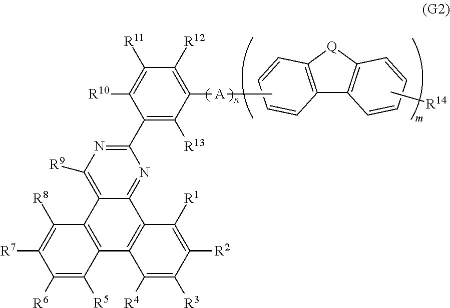

In General Formula (G2), n represents any of 0 to 3, and in represents 1 or 2. Further, A represents a single bond or a substituted or unsubstituted arylene group having 6 to 13 carbon atoms, and Q represents any of S, N, and O. In the case where Q represents N, a substituted or unsubstituted phenyl group may be included as a substituent. Further, each of R¹ to R¹⁴ independently represents any of hydrogen, a substituted or unsubstituted alkyl group having 1 to 6 carbon atoms, a substituted or unsubstituted cycloalkyl group having 5 to 7 carbon atoms, and a substituted or unsubstituted aryl group having 6 to 13 carbon atom.

The term “substitute” in the above structures preferably refers to substitution by a substituent such as an alkyl group having 1 to 6 carbon atoms, e.g., a methyl group, an ethyl group, an n-propyl group, an iso-propyl group, a sec-butyl group, a tert-butyl group, an n-pentyl group, or an n-hexyl group, or substitution by a substituent such as an aryl group having 6 to 12 carbon atoms, e.g., a phenyl group, an o-tolyl group, an m-tolyl group, a p-tolyl group, a 1-napthly group, a 2-naphthyl group, a 2-biphenyl group, a 3-biphenyl group, or a 4-biphenyl group. These substituents may be bonded to each other to form a ring. For example, in the case where the arylene group is a 2,7-fluorenylene group having two phenyl groups at the 9-position as a substituent, the phenyl groups may be bonded to each other to become a spiro-9,9′-bifluorene-2,7-diyl group.

Specific examples of the alkyl group having 1 to 6 carbon atoms in R¹ to R¹⁴ in General Formulae (G1) and (G2) include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a sec-hexyl group, a tert-hexyl group, a neohexyl group, a 3-methylpentyl group, a 2-methylpentyl group, a 2-ethylbutyl group, a 1,2-dimethylbutyl group, and a 2,3-dimethylbutyl group.

Specific examples of the cycloalkyl group having 5 to 7 carbon atoms in R¹ to R¹⁴ in General Formulae (G1) and (G2) include a cycloheptyl group, a cyclohexyl group, and a cycloheptyl group.

Specific examples of the substituted or unsubstituted aryl group having 6 to 13 carbon atoms in R¹ to R¹⁴ in General Formulae (G1) and (G2) include a substituted or unsubstituted phenyl group, a substituted or unsubstituted biphenyl group, a substituted or unsubstituted naphthyl group, a substituted or unsubstituted fluorenyl group, a substituted or unsubstituted phenanthryl group, and a substituted or unsubstituted indenyl group. The term “substitute” in the above structures preferably refers to substitution by a substituent such as an alkyl group having 1 to 6 carbon atoms, e.g., a methyl group, an ethyl group, an n-propyl group, an iso-propyl group, a sec-butyl group, a tert-butyl group, an n-pentyl group, or an n-hexyl group, or substitution by a substituent such as an aryl group having 6 to 12 carbon atoms, e.g., a phenyl group, an o-tolyl group, an m-tolyl group, a p-tolyl group, a 1-napthly group, a 2-naphthyl group, a 2-biphenyl group, a 3-biphenyl group, or a 4-biphenyl group. These substituents may be bonded to each other to form a ring. For example, in the case where the aryl group is a 2-fluorenyl group having two phenyl groups at the 9-position as a substituent, the phenyl groups may be bonded to each other to become a spiro-9,9′-bifluoren-2-yl group. More specifically, a phenyl group, a tolyl group, a xylyl group, a biphenyl group, an indenyl group, a naphthyl group, a fluorenyl group, and the like can be given.

Next, specific structural formulae of the organic compounds of embodiments of the present invention are shown below. Note that the present invention is not limited these to.

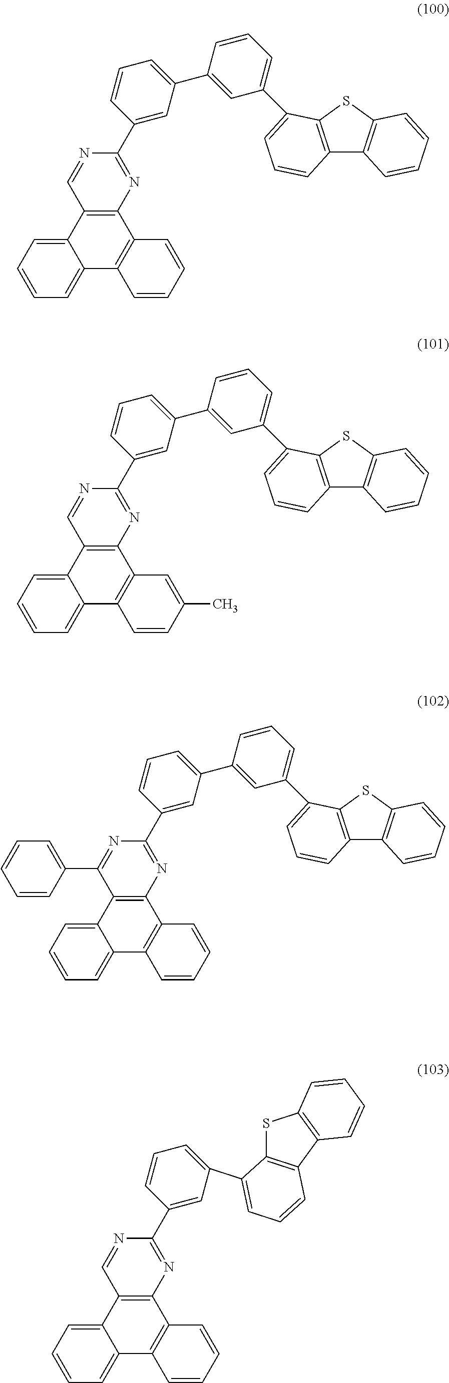 [SMILES: c1cc(-c2cccc(-c3cccc4c3sc3ccccc34)c2)cc(-c2ncc3c4ccccc4c4ccccc4c3n2)c1 ; Cc1ccc2c3ccccc3c3cnc(-c4cccc(-c5cccc(-c6cccc7c6sc6ccccc67)c5)c4)nc3c2c1 ; c1ccc(-c2nc(-c3cccc(-c4cccc(-c5cccc6c5sc5ccccc56)c4)c3)nc3c4ccccc4c4ccccc4c23)cc1 ; c1cc(-c2ncc3c4ccccc4c4ccccc4c3n2)cc(-c2cccc3c2sc2ccccc23)c1]

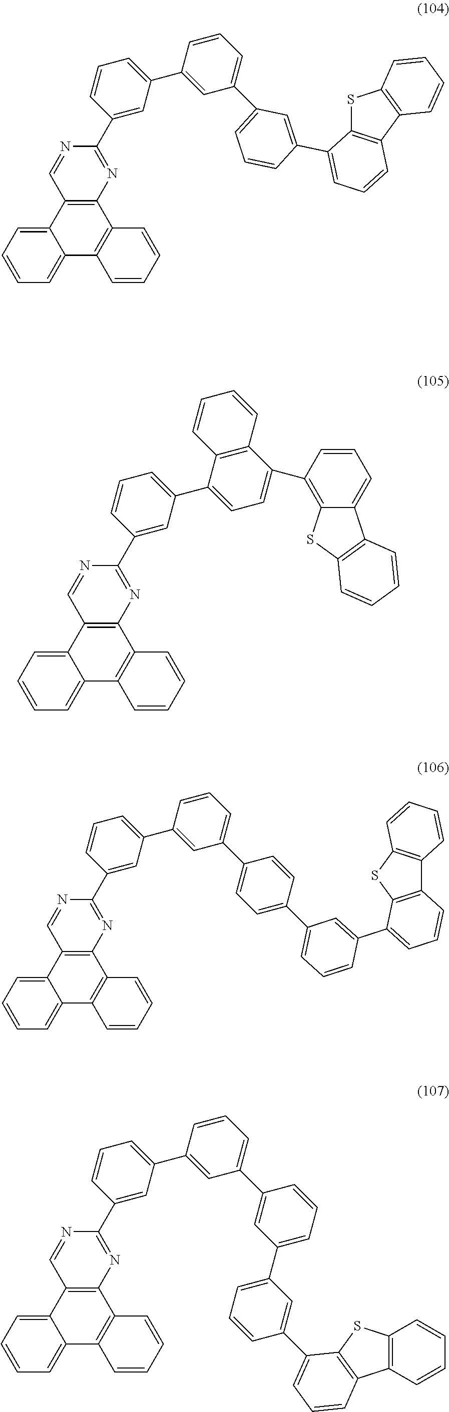 [SMILES: c1cc(-c2cccc(-c3ncc4c5ccccc5c5ccccc5c4n3)c2)cc(-c2cccc(-c3cccc4c3sc3ccccc34)c2)c1 ; c1cc(-c2ncc3c4ccccc4c4ccccc4c3n2)cc(-c2ccc(-c3cccc4c3sc3ccccc34)c3ccccc23)c1 ; c1cc(-c2ccc(-c3cccc(-c4cccc5c4sc4ccccc45)c3)cc2)cc(-c2cccc(-c3ncc4c5ccccc5c5ccccc5c4n3)c2)c1 ; c1cc(-c2cccc(-c3cccc(-c4cccc5c4sc4ccccc45)c3)c2)cc(-c2cccc(-c3ncc4c5ccccc5c5ccccc5c4n3)c2)c1]

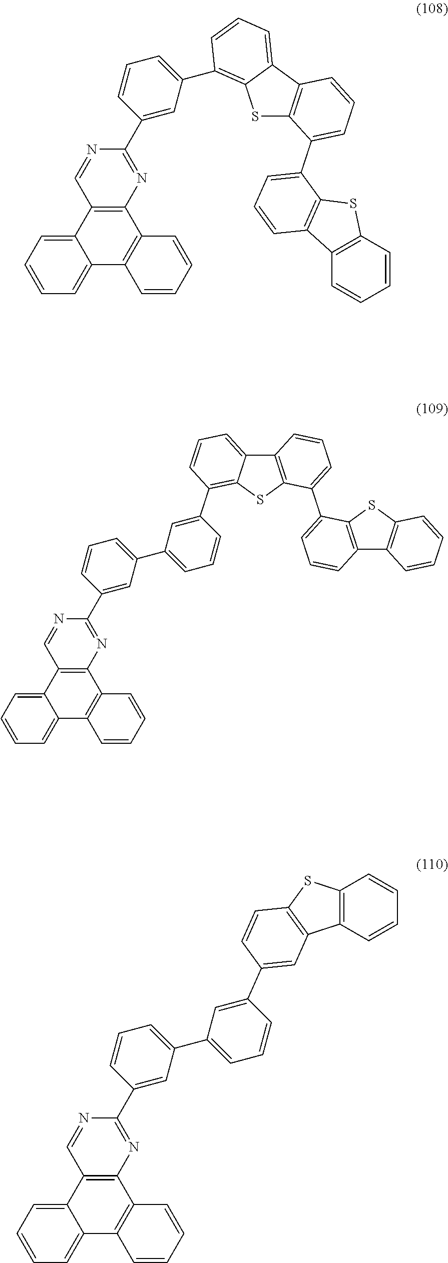 [SMILES: c1cc(-c2ncc3c4ccccc4c4ccccc4c3n2)cc(-c2cccc3c2sc2c(-c4cccc5c4sc4ccccc45)cccc23)c1 ; c1cc(-c2cccc(-c3cccc4c3sc3c(-c5cccc6c5sc5ccccc56)cccc34)c2)cc(-c2ncc3c4ccccc4c4ccccc4c3n2)c1 ; c1cc(-c2cccc(-c3ncc4c5ccccc5c5ccccc5c4n3)c2)cc(-c2ccc3sc4ccccc4c3c2)c1]

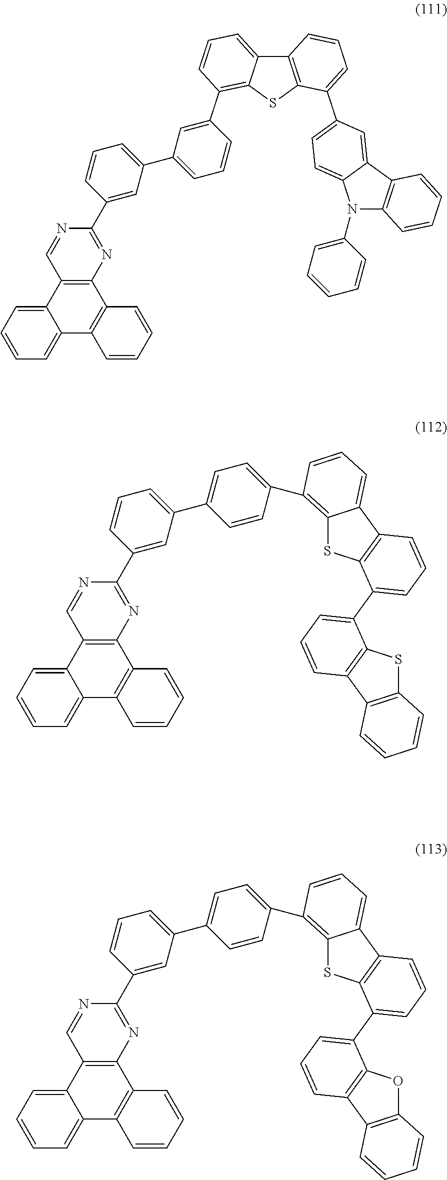 [SMILES: c1ccc(-n2c3ccccc3c3cc(-c4cccc5c4sc4c(-c6cccc(-c7cccc(-c8ncc9c%10ccccc%10c%10ccccc%10c9n8)c7)c6)cccc45)ccc32)cc1 ; c1cc(-c2ccc(-c3cccc4c3sc3c(-c5cccc6c5sc5ccccc56)cccc34)cc2)cc(-c2ncc3c4ccccc4c4ccccc4c3n2)c1 ; c1cc(-c2ccc(-c3cccc4c3sc3c(-c5cccc6c5oc5ccccc56)cccc34)cc2)cc(-c2ncc3c4ccccc4c4ccccc4c3n2)c1]

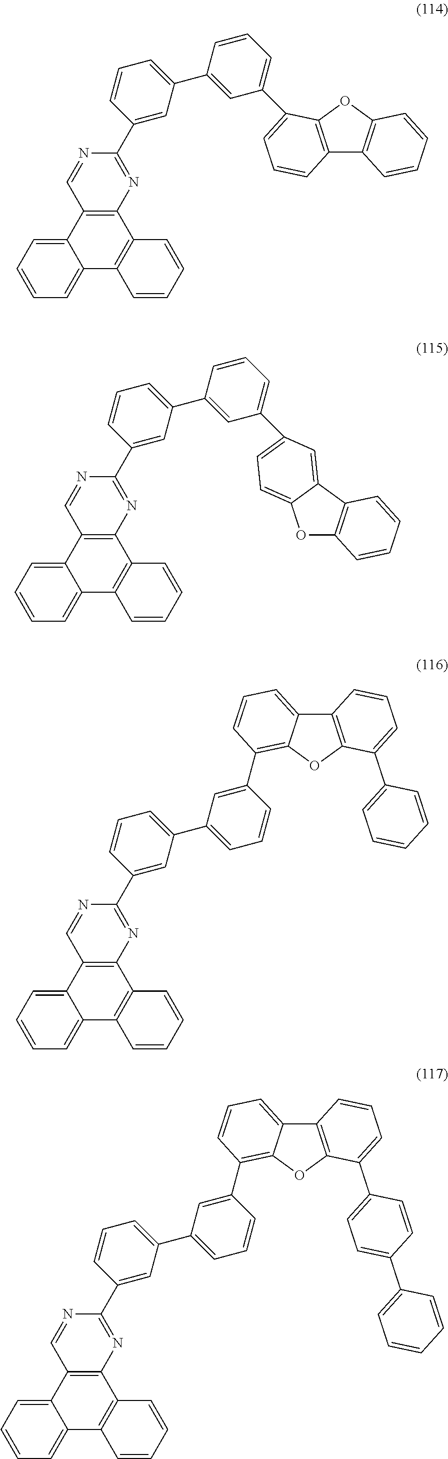 [SMILES: c1cc(-c2cccc(-c3cccc4c3oc3ccccc34)c2)cc(-c2ncc3c4ccccc4c4ccccc4c3n2)c1 ; c1cc(-c2cccc(-c3ncc4c5ccccc5c5ccccc5c4n3)c2)cc(-c2ccc3oc4ccccc4c3c2)c1 ; c1ccc(-c2cccc3c2oc2c(-c4cccc(-c5cccc(-c6ncc7c8ccccc8c8ccccc8c7n6)c5)c4)cccc23)cc1 ; c1ccc(-c2ccc(-c3cccc4c3oc3c(-c5cccc(-c6cccc(-c7ncc8c9ccccc9c9ccccc9c8n7)c6)c5)cccc34)cc2)cc1]

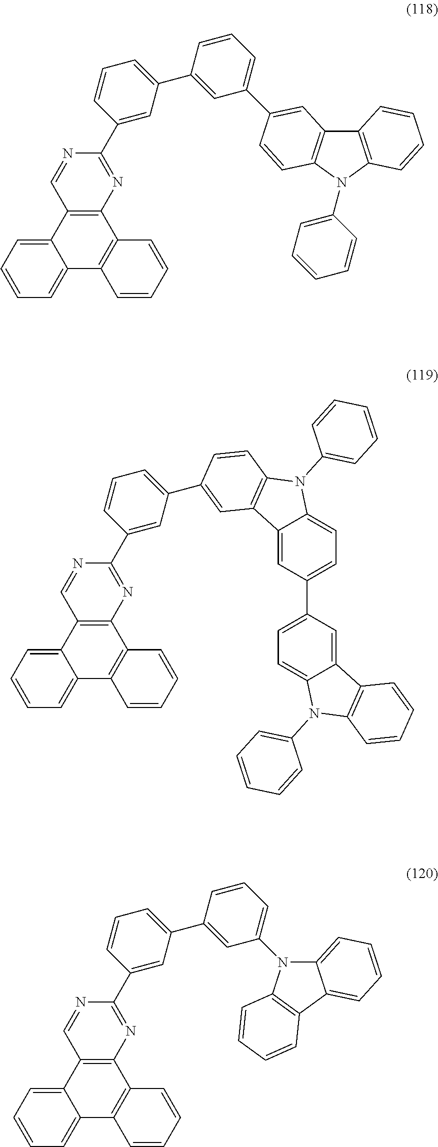 [SMILES: c1ccc(-n2c3ccccc3c3cc(-c4cccc(-c5cccc(-c6ncc7c8ccccc8c8ccccc8c7n6)c5)c4)ccc32)cc1 ; c1ccc(-n2c3ccccc3c3cc(-c4ccc5c(c4)c4cc(-c6cccc(-c7ncc8c9ccccc9c9ccccc9c8n7)c6)ccc4n5-c4ccccc4)ccc32)cc1 ; c1cc(-c2cccc(-n3c4ccccc4c4ccccc43)c2)cc(-c2ncc3c4ccccc4c4ccccc4c3n2)c1]

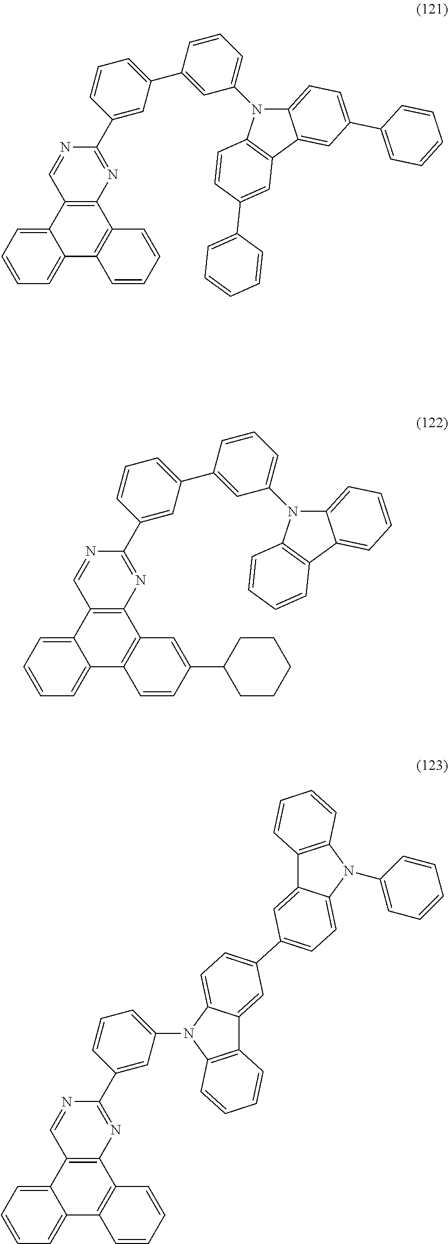 [SMILES: c1ccc(-c2ccc3c(c2)c2cc(-c4ccccc4)ccc2n3-c2cccc(-c3cccc(-c4ncc5c6ccccc6c6ccccc6c5n4)c3)c2)cc1 ; c1cc(-c2cccc(-n3c4ccccc4c4ccccc43)c2)cc(-c2ncc3c4ccccc4c4ccc(C5CCCCC5)cc4c3n2)c1 ; c1ccc(-n2c3ccccc3c3cc(-c4ccc5c(c4)c4ccccc4n5-c4cccc(-c5ncc6c7ccccc7c7ccccc7c6n5)c4)ccc32)cc1]

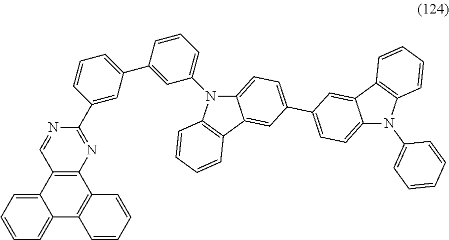 [SMILES: c1ccc(-n2c3ccccc3c3cc(-c4ccc5c(c4)c4ccccc4n5-c4cccc(-c5cccc(-c6ncc7c8ccccc8c8ccccc8c7n6)c5)c4)ccc32)cc1]

Note that the organic compounds represented by Structural Formulae (100) to (124) are examples of the organic compounds represented by General Formulae (G1) and (G2) and an organic compound of one embodiment of the present invention is not limited thereto.

Next, an example of a method for synthesizing the organic compound of one embodiment of the present invention is described.

### <<Method of Synthesizing Heterocyclic Compound Represented by General Formula (G1)>>

First, an example of a method of synthesizing the heterocyclic compound represented by General Formula (G1) is described.

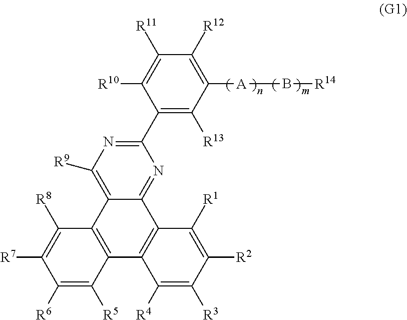

In General Formula (G1), A represents a single bond, or a substituted or unsubstituted arylene group having 6 to 13 carbon atoms; B represents a substituted or unsubstituted dibenzofuran skeleton, a substituted or unsubstituted dibenzothiophene skeleton, or a substituted or unsubstituted carbazole skeleton; and each of R¹ to R¹⁴ independently represents any of hydrogen, a substituted or unsubstituted alkyl group having 1 to 6 carbon atoms, a substituted or unsubstituted cycloalkyl group having 5 to 7 carbon atoms, and a substituted or unsubstituted aryl group having 6 to 13 carbon atoms.

Synthesis Scheme (a) of the heterocyclic compound represented by General Formula (G1) is shown below. Note that the heterocyclic compound represented by General Formula (G1) can be obtained by reaction between a halide (A1) of a dibenzoquinazoline derivative substituted by a phenyl group or a derivative thereof and an arylboronic acid compound (A2) substituted by dibenzothiophene, dibenzofuran, carbazole, or a derivative thereof, as shown in Synthesis Scheme (a).

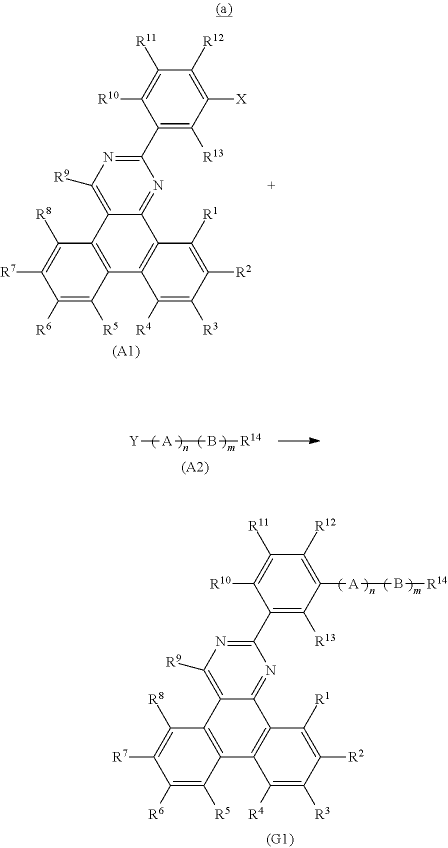

Alternatively, the heterocyclic compound represented by General Formula (G1) can be obtained by reaction between a halide (B1) of a dibenzoquinazoline derivative substituted by an aryl group and a boronic acid compound (B2) of dibenzothiophene, dibenzofuran, carbazole, or a derivative thereof, as shown in Synthesis Scheme (b).

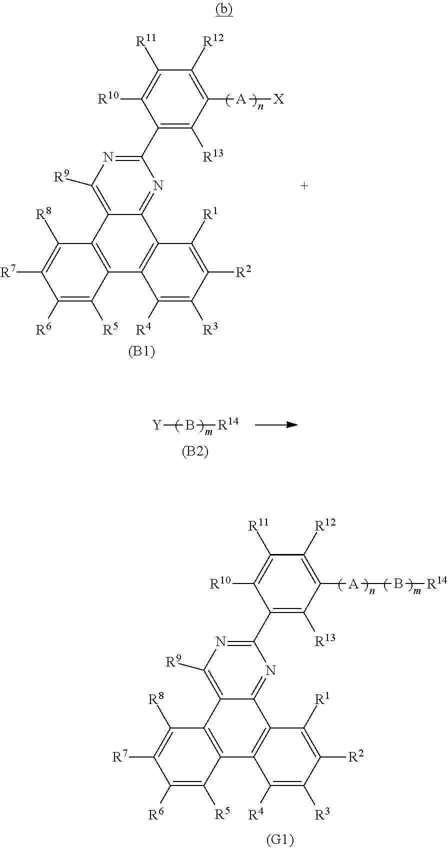

Note that the compound (B1) in Synthesis Scheme (b) can be synthesized as shown in Synthesis Scheme (c). That is, the compound (B1) can be obtained by reaction between a halide (C1) of a dibenzoquinazoline derivative and an arylboronic acid compound (C2). Alternatively, the compound (B1) may be obtained by reaction between a halide (C3) of an arylamidine derivative substituted by phenanthrene or a derivative thereof and N,N′-dimethylformamide diethyl acetal.

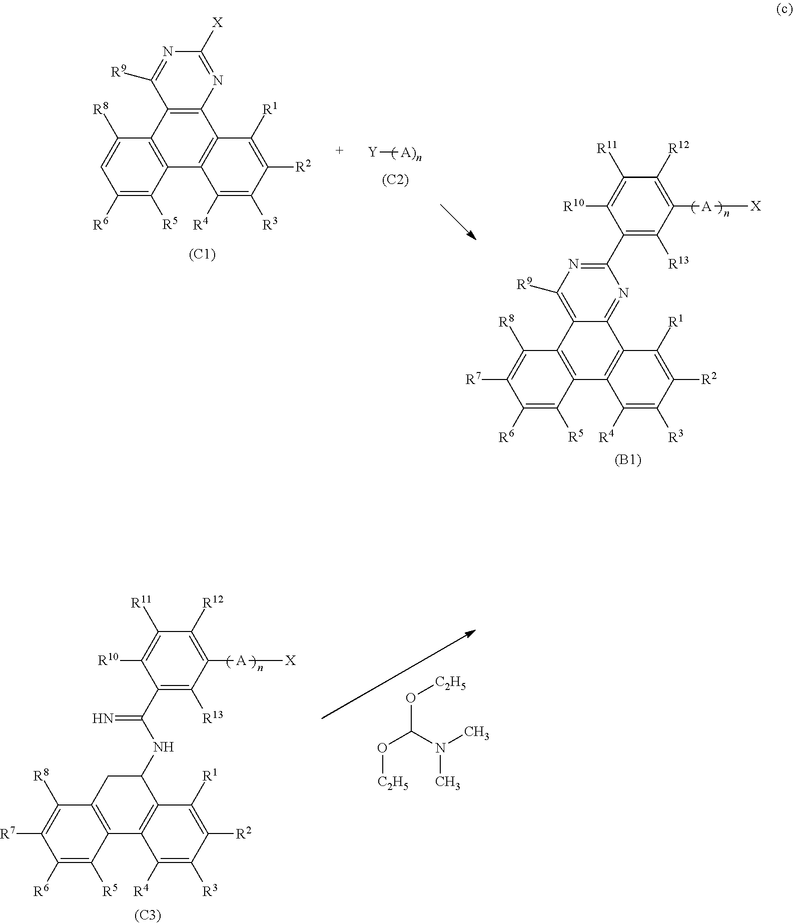

In Synthesis Schemes (a), (b), and (c), each of R¹ to R¹⁴ independently represents any of hydrogen, a substituted or unsubstituted alkyl group having 1 to 6 carbon atoms, a substituted or unsubstituted cycloalkyl group having 5 to 7 carbon atoms, and a substituted or unsubstituted aryl group having 6 to 13 carbon atoms. Further, X represents a halogen element, and is preferably chlorine, bromine, or iodine. Further, Y represents a boronic acid, a boronic ester, a cyclic-triolborate salt, or the like. As the cyclic-triolborate salt, a lithium salt, a potassium salt, or a sodium salt may be used.

Alternatively, the heterocyclic compound represented by General Formula (G1) can be obtained by reaction between a halide of a dibenzoquinazoline derivative substituted by an aryl group and an arylboronic acid compound substituted by dibenzothiophene, dibenzofuran, carbazole, or a derivative thereof, although not shown in a scheme here.

Since various kinds of compounds are available commercially or can be synthesized as the compounds (A1), (A2), (B1), (B2), (C1), (C2), and (C3) in Synthesis Schemes (a) to (c), many kinds of organic compounds can be synthesized as the organic compound represented by General Formula (G1). Thus, a feature of the organic compound which is one embodiment of the present invention is the abundance of variations.

Note that the above-described compound (B1) is a novel compound which is useful in synthesizing the organic compound of one embodiment of the present invention, and is included as one embodiment of the present invention.

The above is the description of the examples of a method of synthesizing the heterocyclic compound of one embodiment of the present invention; however, the present invention is not limited thereto and any other synthesis method may be employed.

Note that the above organic compounds which are embodiments of the present invention each have an electron-transport property and a hole-transport property, and thus can be used as host materials in light-emitting layers, or can be used in electron-transport layers and hole-transport layers. Furthermore, the above organic compounds are materials with a high T1 level, and thus are preferably used in combination with a substance that emits phosphorescence (phosphorescent material) as host materials. In addition, the above organic compounds emit fluorescence and thus can be used as light-emitting substances of light-emitting elements. Accordingly, light-emitting elements containing these organic compounds are also included as embodiments of the present invention.

With the use of the organic compound of one embodiment of the present invention, a light-emitting element, a light-emitting device, an electronic device, or a lighting device having high emission efficiency can be obtained. It is also possible to obtain a light-emitting element, a light-emitting device, an electronic device, or a lighting device with low power consumption.

In Embodiment 1, one embodiment of the present invention has been described. Other embodiments of the present invention will be described in Embodiments 2 to 8. Note that one embodiment of the present invention is not limited thereto. In other words, various embodiments of the invention are described in this embodiment and the other embodiments, and one embodiment of the present invention is not limited to a particular embodiment. For example, although the example in which one embodiment of the present invention is applied to a light-emitting element is described, one embodiment of the present invention is not limited thereto. Depending on circumstances or conditions, one embodiment of the present invention may be applied to objects other than a light-emitting element. Furthermore, depending on circumstances or conditions, one embodiment of the present invention need not be applied to a light-emitting element.

The structure described in this embodiment can be used in appropriate combination with the structure described in any of the other embodiments.

### Embodiment 2

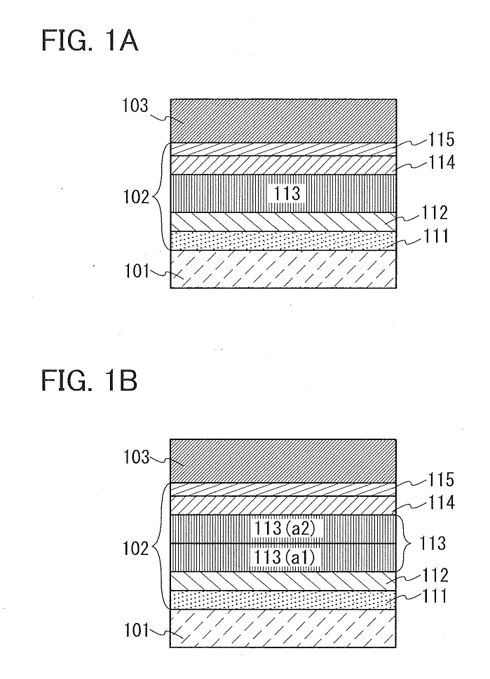

In this embodiment, a light-emitting element in which the organic compound described in Embodiment 1 as one embodiment of the present invention is used is described with reference to FIGS. 1A and 1B.

In the light-emitting element described in this embodiment, an EL layer 102 including a light-emitting layer 113 is provided between a pair of electrodes (a first electrode (anode) 101 and a second electrode (cathode) 103). The EL layer 102 includes, in addition to the light-emitting layer 113, a hole-injection layer 111, a hole-transport layer 112, an electron-transport layer 114, an electron-injection layer 115, and the like.

When a voltage is applied to the light-emitting element, holes injected from the first electrode 101 side and electrons injected from the second electrode 103 side recombine in the light-emitting layer 113; with energy generated by the recombination, a light-emitting substance contained in the light-emitting layer 113 emits light.

A specific example of the light-emitting element described in this embodiment is explained below.

As the first electrode (anode) 101 and the second electrode (cathode) 103, a metal, an alloy, an electrically conductive compound, a mixture thereof, and the like can be used. Specifically, indium oxide-tin oxide (indium tin oxide), indium oxide-tin oxide containing silicon or silicon oxide, indium oxide-zinc oxide (indium zinc oxide), indium oxide containing tungsten oxide and zinc oxide, gold (Au), platinum (Pt), nickel (Ni), tungsten (W), chromium (Cr), molybdenum (Mo), iron (Fe), cobalt (Co), copper (Cu), palladium (Pd), and titanium (Ti) can be used. In addition, an element belonging to Group 1 or Group 2 of the periodic table, for example, an alkali metal such as lithium (Li) or cesium (Cs), an alkaline earth metal such as calcium (Ca) or strontium (Sr), magnesium (Mg), an alloy containing such an element (MgAg, AlLi), a rare earth metal such as europium (Eu) or ytterbium (Yb), an alloy containing such an element, graphene, and the like can be used. The first electrode (anode) 101 and the second electrode (cathode) 103 can be formed by, for example, a sputtering method, an evaporation method (including a vacuum evaporation method), or the like.

As the substance having a high hole-transport property used for the hole-injection layer 111 and the hole-transport layer 112, the following can be given, for example: aromatic amine compounds such as 4,4′-bis[N-(1-naphthyl)-N-phenylamino]biphenyl (abbreviation: NPB or α-NPD), N,N′-bis(3-methylphenyl)-N,N′-diphenyl-[1,1?-biphenyl]-4,4′-diamine (abbreviation: TPD), 4,4′,4″-tris(carbazol-9-yl)triphenylamine (abbreviation: TCTA), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluoren-2-yl)-N-phenylamino]biphenyl (abbreviation: BSPB); 3-[N-(9-phenylcarbazol-3-yl)-N-phenylamino]-9-phenylcarbazole (abbreviation: PCzPCA1); 3,6-bis[N-(9-phenylcarbazol-3-yl)-N-phenylamino]-9-phenylcarbazole (abbreviation: PCzPCA2); 3-[N-(1-naphthyl)-N-(9-phenylcarbazol-3-yl)amino]-9-phenylcarbazole (abbreviation: PCzPCN1); and the like. Alternatively, the following carbazole derivatives can be used: 4,4′-di(N-carbazolyl)biphenyl (abbreviation: CBP), 1,3,5-tris[4-(N-carbazolyephenyl]benzene (abbreviation: TCPB), and 9-[4-(10-phenyl-9-anthracenyl)phenyl]-9H-carbazole (abbreviation: CzPA). Note that the organic compound of one embodiment of the present invention described in Embodiment 1, having a hole-transport property, can also be used. The substances described here are mainly substances having a hole mobility of 1×10⁻⁶ cm²/Vs or higher. However, substances other than the above substances may be used as long as the substance has a higher hole-transport property than an electron-transport property.

Further, a high molecular compound such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), or poly[N,N′-bis(4-butylphenyl)-N,N′-bis(phenyl)benzidine] (abbreviation: Poly-TPD) can be used.

Examples of the acceptor substance that is used for the hole-injection layer 111 include oxides of metals belonging to Groups 4 to 8 of the periodic table. Specifically, molybdenum oxide is particularly preferable.

The light-emitting layer 113 contains a light-emitting substance. Note that the light-emitting layer 113 may contain a first organic compound with an electron-transport property and a hole-transport property (i.e., a host material) and a light-emitting substance (i.e., a guest material). As described in Embodiment 1, the organic compound of one embodiment of the present invention includes a dibenzoquinazoline ring and a skeleton with a hole-transport property. That is, the organic compound of one embodiment of the present invention is a compound with an electron-transport property and a hole-transport property, and therefore is suitable for a host material of a light-emitting layer which is required to allow both of holes and electrons to flow. In particular, a dibenzoquinazoline ring which is stable toward an electron (reduction) accepts electrons and a skeleton with a hole-transport property which is stable toward a hole accepts holes, and the electrons and the holes recombine; accordingly, the organic compound of one embodiment of the present invention is stable toward electrical excitation and can lead to a longer life of a light-emitting element. Furthermore, having high triplet excitation energy and a high LUMO level, a dibenzoquinazoline ring can excite a phosphorescent compound efficiently. Accordingly, a light-emitting element containing the organic compound of one embodiment of the present invention described in Embodiment 1 as a host material and a phosphorescent compound as a guest material in the light-emitting layer can achieve high efficiency. Note that the specific structure of the skeleton with a hole-transport property is as described in Embodiment 1.

In addition to the first organic compound with an electron-transport property and a hole-transport property (host material) and the light-emitting substance (guest material), the light-emitting layer 113 may contain a second organic compound with a hole-transport property. In that case, however, the combination of the first organic compound and the second organic compound need to be capable of forming an exciplex (also referred to as an excited complex) at the time of recombination of carriers (electrons and holes) in the light-emitting layer. When the exciplex is formed in the light-emitting layer, the fluorescence spectrum of the first organic compound and that of the second organic compound are converted into the emission spectrum of the exciplex which is located on a longer wavelength side. Moreover, when the first organic compound and the second organic compound are selected in such a manner that the emission spectrum of the exciplex largely overlaps with the absorption spectrum of a guest material, energy transfer from the singlet excited state can be maximized. Note that also energy transfer of a triplet excited state to the light-emitting substance is considered to occur from the exciplex, not from the host material.

Although the combination of the first organic compound and the second organic compound can be determined such that an exciplex is formed, a combination of a compound which is likely to accept electrons (a compound having an electron-trapping property) and a compound which is likely to accept holes (a compound having a hole-trapping property) is preferably employed. Note that, since it is preferable that the first organic compound be capable of trapping (or transporting) not only electrons but also holes, the organic compound described in Embodiment 1 can be used as the first organic compound. In addition, the second organic compound is preferably a compound that is likely to accept holes.

Examples of the compound which is likely to accept holes include compounds having triarylamine skeletons such as 4-phenyl-4′-(9-phenyl-9H-carbazol-3-yl)triphenylamine (abbreviation: PCBA1BP), 3-[N-(1-naphthyl)-N-(9-phenylcarbazol-3-yl)amino]-9-phenylcarbazole (abbreviation: PCzPCN1), 4,4′,4″-tris[N-(1-naphthyl)-N-phenylamino]triphenylamine (abbreviation: 1′-TNATA), 2,7-bis[N-(4-diphenylaminophenyl)-N-phenylamino]-spiro-9,9′-bifluorene (abbreviation: DPA2SF), N,N′-bis(9-phenylcarbazol-3-yl)-N,N′-diphenylbenzene-1,3-diamine (abbreviation: PCA2B), N-(9,9-dimethyl-2-N′,N′-diphenylamino-9H-fluoren-7-yl)diphenylamine (abbreviation: DPNF), N,N′,N″-triphenyl-N,N′,N″-tris(9-phenylcarbazol-3-yl)benzene-1,3,5-triamine (abbreviation: PCA3B), 2-[N-(9-phenylcarbazol-3-yl)-N-phenylamino]spiro-9,9′-bifluorene (abbreviation: PCASF), 2-[N-(4-diphenylaminophenyl)-N-phenylamino]spiro-9,9′-bifluorene (abbreviation: DPASF), N,N′-bis[4-(carbazol-9-yl)phenyl]-N,N′-diphenyl-9,9-dimethylfluorene-2,7-diamine (abbreviation: YGA2F), 4,4′-bis[N-(3-methylphenyl)-N-phenylamino]biphenyl (abbreviation: TPD), 4,4′-bis[N-(4-diphenylaminophenyl)-N-phenylamino]biphenyl (abbreviation: DPAB), N-(9,9-dimethyl-9H-fluoren-2-yl)-N-{9,9-dimethyl-2-[N′-phenyl-N′-(9,9-dimethyl-9H-fluoren-2-yl)amino]-9H-fluoren-7-yl}phenylamine (abbreviation: DFLADFL), 3-[N-(9-phenylcarbazol-3-yl)-N-phenylamino]-9-phenylcarbazole (abbreviation: PCzPCA1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 4,4′-bis(N-{4-[N′-(3-methylphenyl)-N′-phenylamino]phenyl}-N-phenylamino)biphenyl (abbreviation: DNTPD), 3,6-bis[N-(4-diphenylaminophenyl)-N-(1-naphthyl)amino]-9-phenylcarbazole (abbreviation: PCzTPN2), and 3,6-bis[N-(9-phenylcarbazol-3-yl)-N-phenylamino]-9-phenylcarbazole (abbreviation: PCzPCA2).

The first organic compound and the second organic compound are not limited to the above examples as long as the combination of the first organic compound and the second organic compound can form an exciplex, the emission spectrum of the exciplex overlaps with the absorption spectrum of the light-emitting substance, and the peak of the emission spectrum of the exciplex has a longer wavelength than the peak of the absorption spectrum of the light-emitting substance.

Note that in the case where a compound which easily accepts electrons and a compound which easily accepts holes are used as the first organic compound and the second organic compound, carrier balance can be controlled by the mixture ratio of the compounds. Specifically, the ratio of the first organic compound to the second organic compound is preferably 1:9 to 9:1.

As the materials that can be used as the light-emitting substance in the light-emitting layer 113, a light-emitting substance converting singlet excitation energy into light emission, a light-emitting substance converting triplet excitation energy into light emission, and the like can be used alone or in combination. Described below are examples of the light-emitting substance and the emission center substance.

As an example of the light-emitting substance converting singlet excitation energy into light emission, a substance which emits fluorescence (a fluorescent compound) can be given.

Examples of a substance which emits fluorescence include N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra(tert-butyl)perylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), and 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM).

Examples of the light-emitting substance converting triplet excitation energy into light emission include a substance which emits phosphorescence (a phosphorescent compound) and a thermally activated delayed fluorescent (TADF) material which emits thermally activated delayed fluorescence. Note that “delayed fluorescence” exhibited by the TADF material refers to light emission having the same spectrum as normal fluorescence and an extremely long lifetime. The lifetime is 1×10⁻⁶ seconds or longer, preferably 1×10⁻³ seconds or longer.

Examples of a substance which emits phosphorescence include bis{2-[3′,5′-bis(trifluoromethyl)phenyl]pyridinato-N,C^{2′}}iridium(III) picolinate (abbreviation: [Ir(CF₃ppy)₂(pic)]), bis[2-(4′,6′-difluorophenyl)pyridinato-N,C^{2′}]iridium(III) acetylacetonate (abbreviation: FIracac), tris(2-phenylpyridinato)iridium(III) (abbreviation: [Ir(ppy)₃]), bis(2-phenylpyridinato)iridium(III) acetylacetonate (abbreviation: [Ir(ppy)₂(acac)]), tris(acetylacetonato) (monophenanthroline)terbium(III) (abbreviation: [Tb(acac)₃(Phen)]), bis(benzo[h]quinolinato)iridium(III) acetylacetonate (abbreviation: [Ir(bzq)₂(acac)]), bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), bis(2-phenylbenzothiazolato-N,C^{2′})iridium(III) acetylacetonate (abbreviation: [Ir(bt)₂(acac)]), bis[2-(2′-benzo[4,5-a]thienyl)pyridinato-N,C^{3′}]iridium(III) acetylacetonate (abbreviation: [Ir(btp)₂(acac)]), bis(1-phenylisoquinolinato-N,C^{2′})iridium(III) acetylacetonate (abbreviation: [Ir(piq)₂(acac)]), (acetylacetonato)bis[2,3-bis(4-fluorophenyl)quinoxalinato]iridium(III) (abbreviation: [Ir(Fdpq)₂(acac)]), (acetylacetonato)bis(3,5-dimethyl-2-phenylpyrazinato)iridium(III) (abbreviation: [Ir(mppr-Me)₂(acac)]), (acetylacetonato)bis(5-isopropyl-3-methyl-2-phenylpyrazinato)iridium(III) (abbreviation: [Ir(mppr-iPr)₂(acac)]), (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), (acetylacetonato)bis(6-tert-butyl-4-phenylpyrimidinato)iridium(III) (abbreviation: [Ir(tBuppm)₂(acac)]), (acetylacetonato)bis(4,6-diphenylpyrimidinato)iridium(III) (abbreviation: [Ir(dppm)₂(acac)]), 2,3,7,8,12,13,17,18-octaethyl-21H,23H-porphyrin platinum(II) (abbreviation: PtOEP), tris(1,3-diphenyl-1,3-propanedionato) (monophenanthroline)europium(III) (abbreviation: [Eu(DBM)₃(Phen)]), and tris[1-(2-thenoyl)-3,3,3-trifluoroacetonato] (monophenanthroline)europium(III) (abbreviation: [Eu(TTA)₃(Phen)]).

Examples of the TADF material includes fullerene, a derivative thereof, an acridine derivative such as proflavine, and eosin. Other examples include a metal-containing porphyrin, such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP). Alternatively, a heterocyclic compound including a π-electron rich heteroaromatic ring and a π-electron deficient heteroaromatic ring can be used, such as 2-(biphenyl-4-yl)-4,6-bis(12-phenylindolo[2,3-a]carbazol-11-yl)-1,3,5-triazine (PIC-TRZ). Note that a material in which the π-electron rich heteroaromatic ring is directly bonded to the π-electron deficient heteroaromatic ring is particularly preferably used because both the donor property of the π-electron rich heteroaromatic ring and the acceptor property of the π-electron deficient heteroaromatic ring are increased and the energy difference between the S1 level and the T1 level becomes small.

The light-emitting layer 113 does not necessarily have the single-layer structure shown in FIG. 1A and may have a stacked-layer structure including two or more layers as shown in FIG. 1B. In that case, each layer in the stacked structure emits light. For example, fluorescence is obtained from a first light-emitting layer 113(a1) in the stacked structure, and phosphorescence is obtained from a second light-emitting layer 113(a2) stacked over the first layer. Note that the stacking order may be reversed. It is preferable that light emission due to energy transfer from an exciplex to a dopant be obtained from the layer that emits phosphorescence. In the case where blue light emission is obtained from one of the first and second layers, orange or yellow light emission can be obtained from the other layer. Each layer may contain various kinds of dopants.

The electron-transport layer 114 is a layer containing a substance having a high electron-transport property (also referred to as an electron-transport compound). For the electron-transport layer 114, a metal complex such as tris(8-quinolinolato)aluminum(III) (abbreviation: Alq₃), tris(4-methyl-8-quinolinolato)aluminum(Ill) (abbreviation: Almq₃), bis(10-hydroxybenzo[h]quinolinato)beryllium (abbreviation: BeBq₂), bis(2-methyl-8-quinolinolato)(4-phenylphenolato)aluminum(III) (abbreviation: BAlq), bis[2-(2-hydroxyphenyl)benzoxazolato]zinc(II) (abbreviation: Zn(BOX)₂), or bis[2-(2-hydroxyphenyl)benzothiazolato]zinc(II) (abbreviation: Zn(BTZ)₂) can be used. Alternatively, a heteroaromatic compound such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4′-tert-butylphenyl)-4-phenyl-5-(4″-biphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: Bphen), bathocuproine (abbreviation: BCP), or 4,4′-bis(5-methylbenzoxazol-2-yl)stilbene (abbreviation: BzOs) can also be used. A high molecular compound such as poly(2,5-pyridinediyl) (abbreviation: PPy), poly[(9,9-dihexylfluorene-2,7-diyl)-co-(pyridine-3,5-diyl)] (abbreviation: PF-Py), or poly[(9,9-dioctylfluorene-2,7-diyl)-co-(2,2′-bipyridine-6,6′-diyl)] (abbreviation: PF-BPy) can also be used. The substances listed here are mainly ones that have an electron mobility of 1×10⁻⁶ cm²/Vs or higher. Note that any substance other than the substances listed here may be used for the electron-transport layer 114 as long as the electron-transport property is higher than the hole-transport property.

The electron-transport layer 114 is not limited to a single layer, but may be a stack of two or more layers each containing any of the substances listed above.

The electron-injection layer 115 is a layer containing a substance having a high electron-injection property. For the electron-injection layer 115, an alkali metal, an alkaline earth metal, or a compound thereof, such as lithium fluoride (LiF), cesium fluoride (CsF), calcium fluoride (CaF₂), or lithium oxide (LiOₓ) can be used. A rare earth metal compound like erbium fluoride (ErF₃) can also be used. An electride may also be used for the electron-injection layer 115. Examples of the electride include a substance in which electrons are added at high concentration to calcium oxide-aluminum oxide. Any of the substances for forming the electron-transport layer 114, which are given above, can be used.

A composite material in which an organic compound and an electron donor (donor) are mixed may also be used for the electron-injection layer 115. Such a composite material is excellent in an electron-injection property and an electron-transport property because electrons are generated in the organic compound by the electron donor. In this case, the organic compound is preferably a material that is excellent in transporting the generated electrons. Specifically, the above-listed substances for forming the electron-transport layer 114 (e.g., the metal complexes and heteroaromatic compounds) can be used, for example. As the electron donor, a substance showing an electron-donating property with respect to the organic compound may be used. Specifically, an alkali metal, an alkaline earth metal, and a rare earth metal are preferable, and lithium, cesium, magnesium, calcium, erbium, and ytterbium are given. In addition, an alkali metal oxide or an alkaline earth metal oxide is preferable, and lithium oxide, calcium oxide, barium oxide, and the like are given. A Lewis base such as magnesium oxide can also be used. An organic compound such as tetrathiafulvalene (abbreviation: TTF) can also be used.

Note that each of the hole-injection layer 111, the hole-transport layer 112, the light-emitting layer 113, the electron-transport layer 114, the electron-injection layer 115 can be formed by any one or any combination of the following methods: an evaporation method (including a vacuum evaporation method), a printing method (such as relief printing, intaglio printing, gravure printing, planography printing, and stencil printing), an ink-jet method, a coating method, and the like.

Light emission obtained in the EL layer 102 of the above-described light-emitting element is extracted to the outside through one or both of the first electrode 101 and the second electrode 103. Thus, one or both of the first electrode 101 and the second electrode 103 are electrodes having light-transmitting properties.

The light-emitting element described above can contain the organic compound of one embodiment of the present invention in the EL layer, and thus can achieve high efficiency.

The structure described in this embodiment can be used in appropriate combination with the structure described in any of other embodiments.

### Embodiment 3

Described in this embodiment is a light-emitting element (hereinafter referred to as a tandem light-emitting element) with a structure in which the organic compound of one embodiment of the present invention is used as an EL material in an EL layer and a charge-generation layer is provided between a plurality of EL layers.

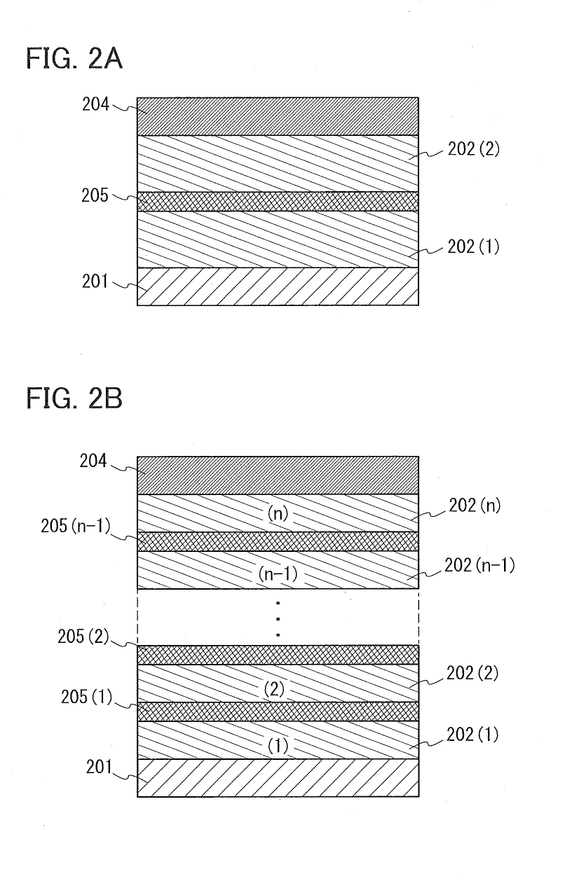

A light-emitting element described in this embodiment is a tandem light-emitting element including a plurality of EL layers (a first EL layer 202(1) and a second EL layer 202(2)) between a pair of electrodes (a first electrode 201 and a second electrode 204), as illustrated in FIG. 2A.

In this embodiment, the first electrode 201 functions as an anode, and the second electrode 204 functions as a cathode. Note that the first electrode 201 and the second electrode 204 can have structures similar to those described in Embodiment 2. In addition, either or both of the EL layers (the first EL layer 202(1) and the second EL layer 202(2)) may have structures similar to those described in Embodiment 2. In other words, the structures of the first EL layer 202(1) and the second EL layer 202(2) may be the same or different from each other and can be similar to those of the EL layers described in Embodiment 2.

In addition, a charge-generation layer 205 is provided between the plurality of EL layers (the first EL layer 202(1) and the second EL layer 202(2)). The charge-generation layer 205 has a function of injecting electrons into one of the EL layers and injecting holes into the other of the EL layers when voltage is applied between the first electrode 201 and the second electrode 204. In this embodiment, when voltage is applied such that the potential of the first electrode 201 is higher than that of the second electrode 204, the charge-generation layer 205 injects electrons into the first EL layer 202(1) and injects holes into the second EL layer 202(2).

Note that in terms of light extraction efficiency, the charge-generation layer 205 preferably has a property of transmitting visible light (specifically, the charge-generation layer 205 has a visible light transmittance of 40% or more). The charge-generation layer 205 functions even when it has lower conductivity than the first electrode 201 or the second electrode 204.

The charge-generation layer 205 may have either a structure in which an electron acceptor (acceptor) is added to an organic compound having a high hole-transport property or a structure in which an electron donor (donor) is added to an organic compound having a high electron-transport property. Alternatively, both of these structures may be stacked.

In the case where an electron acceptor is added to an organic compound having a high hole-transport property, as the organic compound having a high hole-transport property, for example, an aromatic amine compound such as NPB, TPD, TDATA, MTDATA, or BSPB, or the like can be used. The substances listed here are mainly ones that have a hole mobility of 1×10⁻⁶ cm²/Vs or higher. Note that any organic compound other than the compounds listed here may be used as long as the hole-transport property is higher than the electron-transport property.

As the electron acceptor, 7,7,8,8-tetracyano-2,3,5,6-tetrafluoroquinodimethane (abbreviation: F₄-TCNQ), chloranil, and the like can be given. Oxides of metals belonging to Groups 4 to 8 of the periodic table can also be given. Specifically, vanadium oxide, niobium oxide, tantalum oxide, chromium oxide, molybdenum oxide, tungsten oxide, manganese oxide, and rhenium oxide are preferable because of their high electron-accepting properties. Among these, molybdenum oxide is especially preferable because it is stable in the air, has a low hygroscopic property, and is easy to handle.

In the case where an electron donor is added to an organic compound having a high electron-transport property, as the organic compound having a high electron-transport property, for example, a metal complex having a quinoline skeleton or a benzoquinoline skeleton, such as Alq, Almq₃, BeBq₂, and BAlq can be used. Alternatively, a metal complex having an oxazole-based ligand or a thiazole-based ligand, such as Zn(BOX)₂ or Zn(BTZ)₂ can be used. Alternatively, in addition to such a metal complex, PBD, OXD-7, TAZ, Bphen, BCP, and the like can be used. The substances listed here are mainly ones that have an electron mobility of 1×10⁻⁶ cm²/Vs or higher. Note that any organic compound other than the compounds listed here may be used as long as the electron-transport property is higher than the hole-transport property.

As the electron donor, it is possible to use an alkali metal, an alkaline earth metal, a rare earth metal, metals belonging to Groups 2 and 13 of the periodic table, or an oxide or carbonate thereof. Specifically, lithium (Li), cesium (Cs), magnesium (Mg), calcium (Ca), ytterbium (Yb), indium (In), lithium oxide, cesium carbonate, or the like is preferably used. Alternatively, an organic compound such as tetrathianaphthacene may be used as the electron donor.

Note that forming the charge-generation layer 205 by using any of the above materials can suppress a drive voltage increase caused by the stack of the EL layers. The charge-generation layer 205 can be formed by any one or any combination of the following methods: an evaporation method (including a vacuum evaporation method), a printing method (such as relief printing, intaglio printing, gravure printing, planography printing, and stencil printing), an ink-jet method, a coating method, and the like.

Although the light-emitting element including two EL layers is described in this embodiment, the present invention can be similarly applied to a light-emitting element in which n EL layers (202(1) to 202(n)) (n is three or more) are stacked as illustrated in FIG. 2B. In the case where a plurality of EL layers are included between a pair of electrodes as in the light-emitting element according to this embodiment, by providing charge-generation layers (205(1) to 205(n−1)) between the EL layers, light emission in a high luminance region can be obtained with current density kept low. Since the current density can be kept low, the element can have a long lifetime.

When the EL layers have different emission colors, a desired emission color can be obtained from the whole light-emitting element. For example, in a light-emitting element having two EL layers, when an emission color of the first EL layer and an emission color of the second EL layer are complementary colors, the light-emitting element can emit white light as a whole. Note that “complementary colors” refer to colors that can produce an achromatic color when mixed. In other words, mixing light of complementary colors allows white emission to be obtained. Specifically, a combination in which blue light emission is obtained from the first EL layer and yellow light emission or orange light emission is obtained from the second EL layer is given as an example. In that case, it is not necessary that both of blue light emission and yellow (or orange) light emission are fluorescence, and the both are not necessarily phosphorescence. For example, a combination in which blue light emission is fluorescence and yellow (or orange) light emission is phosphorescence or a combination in which blue light emission is phosphorescence and yellow (or orange) light emission is fluorescence may be employed.

The same can be applied to a light-emitting element having three EL layers. For example, the light-emitting element as a whole can provide white light emission when the emission color of the first EL layer is red, the emission color of the second EL layer is green, and the emission color of the third EL layer is blue.

Note that the structure described in this embodiment can be combined as appropriate with any of the structures described in other embodiments.

### Embodiment 4

Described in this embodiment is a light-emitting device that includes a light-emitting element in which the organic compound of one embodiment of the present invention is used for an EL layer.

The light-emitting device may be either a passive matrix light-emitting device or an active matrix light-emitting device. Any of the light-emitting elements described in other embodiments can be used for the light-emitting device described in this embodiment.

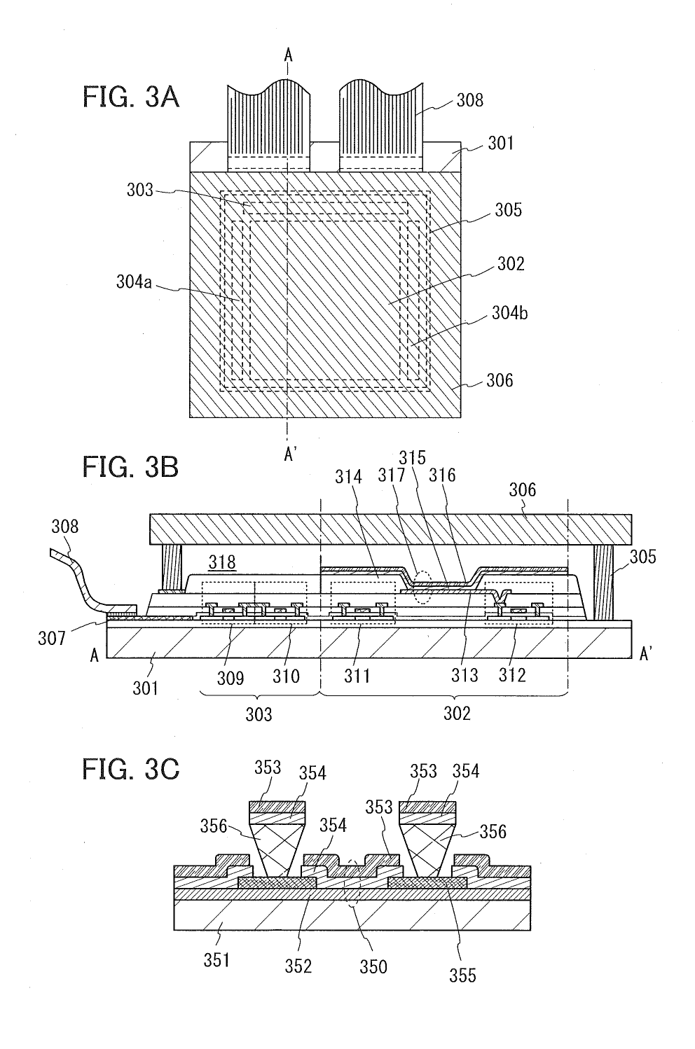

In this embodiment, first, an active matrix light-emitting device is described with reference to FIGS. 3A to 3C.

Note that FIG. 3A is a top view illustrating a light-emitting device and FIG. 3B is a cross-sectional view taken along the chain line A-A? in FIG. 3A. The light-emitting device includes a pixel portion 302 provided over an element substrate 301, a driver circuit portion (a source line driver circuit) 303, and driver circuit portions (gate line driver circuits) 304 (304a and 304b). The pixel portion 302, the driver circuit portion 303, and the driver circuit portions 304 are sealed between the element substrate 301 and a sealing substrate 306 with a sealant 305.

In addition, over the element substrate 301, a lead wiring 307 for connecting an external input terminal, through which a signal (e.g., a video signal, a clock signal, a start signal, a reset signal, or the like) or electric potential from the outside is transmitted to the driver circuit portion 303 and the driver circuit portions 304, is provided. Here, an example is described in which a flexible printed circuit (FPC) 308 is provided as the external input terminal. Although only the FPC is illustrated here, the FPC may be provided with a printed wiring board (PWB). The light-emitting device in this specification includes, in its category, not only the light-emitting device itself but also the light-emitting device provided with the FPC or the PWB.

Next, a cross-sectional structure is described with reference to FIG. 3B. The driver circuit portions and the pixel portion are formed over the element substrate 301; the driver circuit portion 303 that is the source line driver circuit and the pixel portion 302 are illustrated here.

The driver circuit portion 303 is an example in which an FET 309 and an FET 310 are combined. Note that the driver circuit portion 303 may be formed with a circuit including transistors having the same conductivity type (either n-channel transistors or p-channel transistors) or a CMOS circuit including an n-channel transistor and a p-channel transistor. Although this embodiment shows a driver integrated type in which the driver circuit is formed over the substrate, the driver circuit is not necessarily formed over the substrate, and may be formed outside the substrate.

The pixel portion 302 includes a plurality of pixels each of which includes a switching FET 311, a current control FET 312, and a first electrode (anode) 313 which is electrically connected to a wiring (a source electrode or a drain electrode) of the current control FET 312. Although the pixel portion 302 includes two FETs, the switching FET 311 and the current control FET 312, in this embodiment, one embodiment of the present invention is not limited thereto. The pixel portion 302 may include, for example, three or more FETs and a capacitor in combination.

As the FETs 309, 310, 311, and 312, for example, a staggered transistor or an inverted staggered transistor can be used. Examples of a semiconductor material that can be used for the FETs 309, 310, 311, and 312 include a Group 13 semiconductor, a Group 14 semiconductor (e.g., silicon), a compound semiconductor, an oxide semiconductor, and an organic semiconductor material. In addition, there is no particular limitation on the crystallinity of the semiconductor material, and an amorphous semiconductor film or a crystalline semiconductor film can be used. In particular, an oxide semiconductor is preferably used for the FETs 309, 310, 311, and 312. Examples of the oxide semiconductor include an In—Ga oxide and an In-M-Zn oxide (M is Al, Ga, Y, Zr, La, Ce, or Nd). For example, an oxide semiconductor material that has an energy gap of 2 eV or more, preferably 2.5 eV or more, further preferably 3 eV or more is used for the FETs 309, 310, 311, and 312, so that the off-state current of the transistors can be reduced.

In addition, an insulator 314 is formed to cover end portions of the first electrode (anode) 313. In this embodiment, the insulator 314 is faulted using a positive photosensitive acrylic resin. The first electrode 313 is used as an anode in this embodiment.

The insulator 314 preferably has a curved surface with curvature at an upper end portion or a lower end portion thereof. This enables the coverage with a film to be formed over the insulator 314 to be favorable. The insulator 314 can be formed using, for example, either a negative photosensitive resin or a positive photosensitive resin. The material for the insulator 314 is not limited to an organic compound and an inorganic compound such as silicon oxide, silicon oxynitride, or silicon nitride can also be used.

The light-emitting element 317 has a stacked-layer structure including the first electrode (anode) 313, an EL layer 315, and a second electrode (cathode) 316, and the EL layer 315 includes at least a light-emitting layer. In the EL layer 315, a hole-injection layer, a hole-transport layer, an electron-transport layer, an electron-injection layer, a charge-generation layer, and the like can be provided as appropriate in addition to the light-emitting layer.

For the first electrode (anode) 313, the EL layer 315, and the second electrode (cathode) 316, any of the materials given in Embodiment 2 can be used. Although not illustrated, the second electrode (cathode) 316 is electrically connected to the FPC 308 which is an external input terminal.

Although the cross-sectional view in FIG. 3B illustrates only one light-emitting element 317, a plurality of light-emitting elements are arranged in a matrix in the pixel portion 302. Light-emitting elements that emit light of three kinds of colors (R, G, and B) are selectively formed in the pixel portion 302, whereby a light-emitting device capable of full color display can be obtained. In addition to the light-emitting elements that emit light of three kinds of colors (R, G, and B), for example, light-emitting elements that emit light of white (W), yellow (Y), magenta (M), cyan (C), and the like may be formed. For example, the light-emitting elements that emit light of a plurality of kinds of colors are used in combination with the light-emitting elements that emit light of three kinds of colors (R, G, and B), whereby effects such as an improvement in color purity and a reduction in power consumption can be achieved. Alternatively, the light-emitting device may be capable of full color display by combination with color filters. The light-emitting device may have improved emission efficiency and reduced power consumption by combination with quantum dots.

Furthermore, the sealing substrate 306 is attached to the element substrate 301 with the sealant 305, whereby a light-emitting element 317 is provided in a space 318 surrounded by the element substrate 301, the sealing substrate 306, and the sealant 305. Note that the space 318 may be filled with an inert gas (such as nitrogen and argon) or the sealant 305. In the case where the sealant is applied for attachment of the substrates, one or more of UV treatment, heat treatment, and the like are preferably performed.

An epoxy-based resin or glass frit is preferably used for the sealant 305. The material preferably allows as little moisture and oxygen as possible to penetrate. As the sealing substrate 306, a glass substrate, a quartz substrate, or a plastic substrate formed of fiber-reinforced plastic (FRP), poly(vinyl fluoride) (PVF), polyester, acrylic, or the like can be used. In the case where glass frit is used as the sealant, the element substrate 301 and the sealing substrate 306 are preferably glass substrates for high adhesion.

As described above, an active matrix light-emitting device can be obtained.

The light-emitting device including the light-emitting element in which the organic compound of one embodiment of the present invention is contained in the EL layer may be of the passive matrix type, instead of the active matrix type described above.

FIG. 3C is a cross-sectional view illustrating a pixel portion of a passive-matrix light-emitting device.

As illustrated in FIG. 3C, a light-emitting element 350 including a first electrode 352, an EL layer 354, and a second electrode 353 is formed over a substrate 351. Note that the first electrode 352 has an island-like shape, and a plurality of the first electrodes 352 are formed in one direction to form a striped pattern. An insulating film 355 is formed over part of the first electrode 352.

A partition 356 formed using an insulating material is provided over the insulating film 355. The sidewalls of the partition 356 slope so that the distance between one sidewall and the other sidewall gradually decreases toward the surface of the substrate. In other words, a cross section taken along the direction of the short side of the partition 356 is trapezoidal, and the base (a side which is in the same direction as a plane direction of the insulating film 355 and in contact with the insulating film 355) is shorter than the upper side (a side which is in the same direction as the plane direction of the insulating film 355 and not in contact with the insulating film 355). By providing the partition 356 in such a manner, a defect of the light-emitting element due to static electricity or the like can be prevented. Note that the insulating film 355 has an opening portion over part of the first electrode 352, and when the EL layer 354 is formed after formation of the partition 356, the EL layer 354 that is in contact with the first electrode 352 in the opening portion is formed.

After formation of the EL layer 354, the second electrode 353 is formed. Thus, the second electrode 353 is formed over the EL layer 354 and in some cases, is formed over the insulating film 355 without contact with the first electrode 352. Note that since the EL layer 354 and the second electrode 353 are formed after formation of the partition 356, the EL layer 354 and the second electrode 353 are also stacked over the partition 356 sequentially.

Note that sealing can be performed by a method similar to that used for the active matrix light-emitting device, and description thereof is not made.

As described above, a passive matrix light-emitting device can be obtained.

Note that in this specification and the like, a transistor or a light-emitting element can be formed using any of a variety of substrates, for example. The type of a substrate is not limited to a certain type. As the substrate, a semiconductor substrate (e.g., a single crystal substrate or a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, a base material film, or the like can be used, for example. As an example of a glass substrate, a barium borosilicate glass substrate, an aluminoborosilicate glass substrate, a soda lime glass substrate, or the like can be given. Examples of the flexible substrate, the attachment film, the base film, and the like are substrates of plastics typified by polyethylene terephthalate (PET), polyethylene naphthalate (PEN), polyether sulfone (PES), and polytetrafluoroethylene (PTFE). Another example is a synthetic resin such as acrylic. Alternatively, polypropylene, polyester, polyvinyl fluoride, polyvinyl chloride, or the like can be used. Alternatively, polyamide, polyimide, aramid, epoxy, an inorganic vapor deposition film, paper, or the like can be used. Specifically, the use of semiconductor substrates, single crystal substrates, SOI substrates, or the like enables the manufacture of small-sized transistors with a small variation in characteristics, size, shape, or the like and with high current supply capability. A circuit using such transistors achieves lower power consumption of the circuit or higher integration of the circuit.

Alternatively, a flexible substrate may be used as the substrate, and the transistor or the light-emitting element may be provided directly on the flexible substrate. Still alternatively, a separation layer may be provided between the substrate and the transistor or the light-emitting element. The separation layer can be used when part or the whole of a semiconductor device formed over the separation layer is separated from the substrate and transferred onto another substrate. In such a case, the transistor or light-emitting element can be transferred to a substrate having low heat resistance or a flexible substrate. For the separation layer, a stack including inorganic films, which are a tungsten film and a silicon oxide film, or an organic resin film of polyimide or the like formed over a substrate can be used, for example.

In other words, a transistor or a light-emitting element may be formed using one substrate, and then transferred to another substrate. Examples of a substrate to which a transistor or a light-emitting element is transferred include, in addition to the above-described substrates over which transistors and light-emitting elements can be formed, a paper substrate, a cellophane substrate, an aramid film substrate, a polyimide film substrate, a stone substrate, a wood substrate, a cloth substrate (including a natural fiber (e.g., silk, cotton, or hemp), a synthetic fiber (e.g., nylon, polyurethane, or polyester), a regenerated fiber (e.g., acetate, cupra, rayon, or regenerated polyester), or the like), a leather substrate, and a rubber substrate. When such a substrate is used, a transistor with excellent characteristics or low power consumption can be formed, a device with high durability or high heat resistance can be provided, or a reduction in weight or thickness can be achieved.

Note that the structure described in this embodiment can be combined as appropriate with any of the structures described in other embodiments.

### Embodiment 5

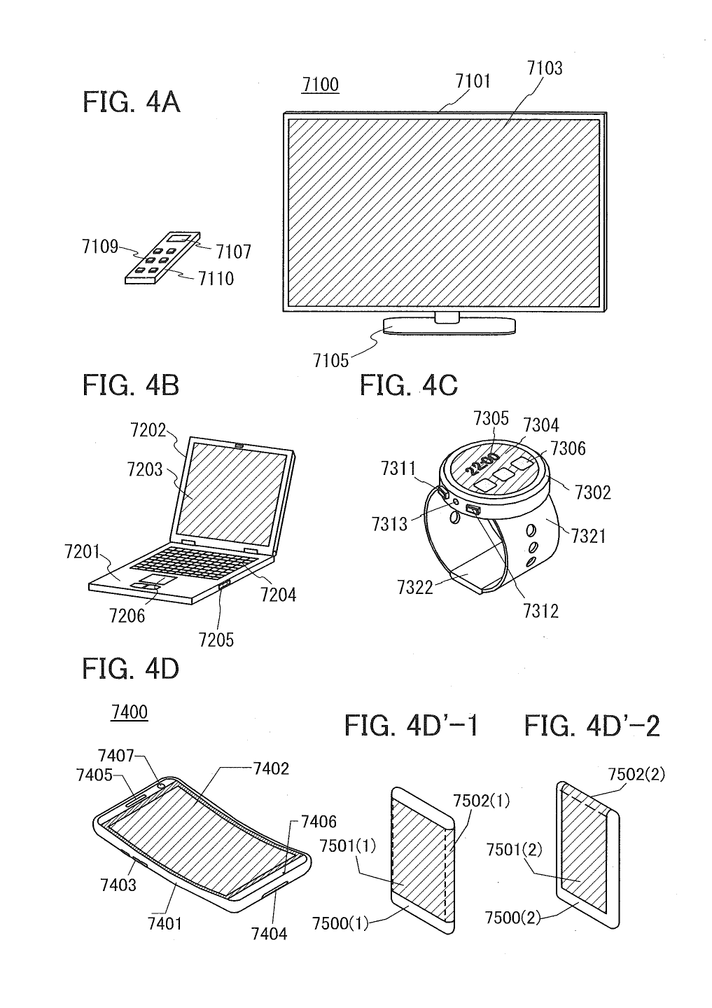

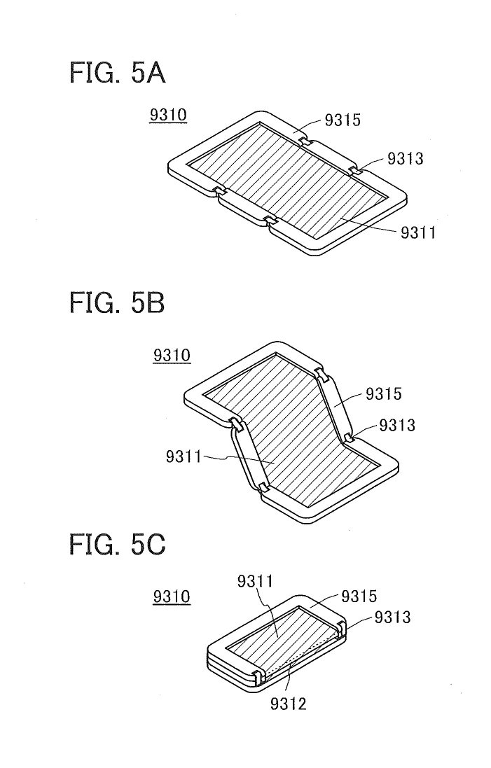

In this embodiment, examples of an electronic device manufactured using a light-emitting device which is one embodiment of the present invention are described with reference to FIGS. 4A to 4D, 4D′-1, and 4D′-2 and FIGS. 5A to 5C.

Examples of the electronic device including the light-emitting device are television devices (also referred to as TV or television receivers), monitors for computers and the like, cameras such as digital cameras and digital video cameras, digital photo frames, cellular phones (also referred to as portable telephone devices), portable game consoles, portable information terminals, audio playback devices, large game machines such as pachinko machines, and the like. Specific examples of the electronic devices are illustrated in FIGS. 4A to 4D, 4D′-1, and 4D′-2.

FIG. 4A illustrates an example of a television device. In the television device 7100, a display portion 7103 is incorporated in a housing 7101. The display portion 7103 can display images and may be a touch panel (an input/output device) including a touch sensor (an input device). Note that the light-emitting device which is one embodiment of the present invention can be used for the display portion 7103. In addition, here, the housing 7101 is supported by a stand 7105.

The television device 7100 can be operated by an operation switch of the housing 7101 or a separate remote controller 7110. With operation keys 7109 of the remote controller 7110, channels and volume can be controlled and images displayed on the display portion 7103 can be controlled. Furthermore, the remote controller 7110 may be provided with a display portion 7107 for displaying data output from the remote controller 7110.

Note that the television device 7100 is provided with a receiver, a modem, and the like. With the use of the receiver, general television broadcasts can be received. Moreover, when the television device is connected to a communication network with or without wires via the modem, one-way (from a sender to a receiver) or two-way (between a sender and a receiver or between receivers) information communication can be performed.

FIG. 4B illustrates a computer, which includes a main body 7201, a housing 7202, a display portion 7203, a keyboard 7204, an external connection port 7205, a pointing device 7206, and the like. Note that this computer can be manufactured using the light-emitting device which is one embodiment of the present invention for the display portion 7203. The display portion 7203 may be a touch panel (an input/output device) including a touch sensor (an input device).

FIG. 4C illustrates a smart watch, which includes a housing 7302, a display portion 7304, operation buttons 7311 and 7312, a connection terminal 7313, a band 7321, a clasp 7322, and the like.

The display portion 7304 mounted in the housing 7302 serving as a bezel includes a non-rectangular display region. The display portion 7304 can display an icon 7305 indicating time, another icon 7306, and the like. The display portion 7304 may be a touch panel (an input/output device) including a touch sensor (an input device).

The smart watch illustrated in FIG. 4C can have a variety of functions, such as a function of displaying a variety of information (e.g., a still image, a moving image, and a text image) on a display portion, a touch panel function, a function of displaying a calendar, date, time, and the like, a function of controlling processing with a variety of software (programs), a wireless communication function, a function of being connected to a variety of computer networks with a wireless communication function, a function of transmitting and receiving a variety of data with a wireless communication function, and a function of reading program or data stored in a recording medium and displaying the program or data on a display portion.

The housing 7302 can include a speaker, a sensor (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone, and the like. Note that the smart watch can be manufactured using the light-emitting device for the display portion 7304.

FIGS. 4D, 4D′-1, and 4D′-2 illustrate an example of a cellular phone (e.g., smartphone). A cellular phone 7400 includes a housing 7401 provided with a display portion 7402, a microphone 7406, a speaker 7405, a camera 7407, an external connection portion 7404, an operation button 7403, and the like. In the case where a light-emitting device is manufactured by forming a light-emitting element of one embodiment of the present invention over a flexible substrate, the light-emitting element can be used for the display portion 7402 having a curved surface as illustrated in FIG. 4D.

When the display portion 7402 of the cellular phone 7400 illustrated in FIG. 4D is touched with a finger or the like, data can be input to the cellular phone 7400. In addition, operations such as making a call and composing e-mail can be performed by touch on the display portion 7402 with a finger or the like.

There are mainly three screen modes of the display portion 7402. The first mode is a display mode mainly for displaying an image. The second mode is an input mode mainly for inputting data such as characters. The third mode is a display-and-input mode in which two modes of the display mode and the input mode are combined.

For example, for making a call or creating e-mail, a character input mode mainly for inputting characters is selected for the display portion 7402 so that characters displayed on the screen can be input. In this case, it is preferable to display a keyboard or number buttons on almost the entire screen of the display portion 7402.

When a detection device such as a gyroscope or an acceleration sensor is provided inside the cellular phone 7400, display on the screen of the display portion 7402 can be automatically changed by determining the orientation of the cellular phone 7400 (whether the cellular phone is placed horizontally or vertically for a landscape mode or a portrait mode).

The screen modes are changed by touch on the display portion 7402 or operation with the operation button 7403 of the housing 7401. The screen modes can be switched depending on the kind of images displayed on the display portion 7402. For example, when a signal of an image displayed on the display portion is a signal of moving image data, the screen mode is switched to the display mode. When the signal is a signal of text data, the screen mode is switched to the input mode.

Moreover, in the input mode, if a signal detected by an optical sensor in the display portion 7402 is detected and the input by touch on the display portion 7402 is not performed for a certain period, the screen mode may be controlled so as to be changed from the input mode to the display mode.

The display portion 7402 may function as an image sensor. For example, an image of a palm print, a fingerprint, or the like is taken by touch on the display portion 7402 with the palm or the finger, whereby personal authentication can be performed. In addition, by providing a backlight or a sensing light source that emits near-infrared light in the display portion, an image of a finger vein, a palm vein, or the like can be taken.

The light-emitting device can be used for a cellular phone having a structure illustrated in FIG. 4D′-1 or FIG. 4D′-2, which is another structure of the cellular phone (e.g., smartphone).

Note that with the structure illustrated in FIG. 4D′-1 or FIG. 4D′-2, text data, image data, or the like can be displayed on second screens 7502(1) and 7502(2) of housings 7500(1) and 7500(2) as well as first screens 7501(1) and 7501(2). Such a structure enables a user to easily see text data, image data, or the like displayed on the second screens 7502(1) and 7502(2) while the cellular phone is placed in user's breast pocket.

FIGS. 5A to 5C illustrate a foldable portable information terminal 9310. FIG. 5A illustrates the portable information terminal 9310 which is opened. FIG. 5B illustrates the portable information terminal 9310 which is being opened or being folded. FIG. 5C illustrates the portable information terminal 9310 that is folded. The portable information terminal 9310 is highly portable when folded. The portable information terminal 9310 is highly browsable when opened because of a seamless large display region.

A display portion 9311 is supported by three housings 9315 joined together by hinges 9313. Note that the display portion 9311 may be a touch panel (an input/output device) including a touch sensor (an input device). By bending the display portion 9311 at a connection portion between two housings 9315 with the use of the hinges 9313, the portable information terminal 9310 can be reversibly changed in shape from an opened state to a folded state. A light-emitting device of one embodiment of the present invention can be used for the display portion 9311. A display region 9312 in the display portion 9311 is a display region that is positioned at a side surface of the portable information terminal 9310 that is folded. On the display region 9312, information icons, file shortcuts of frequently used applications or programs, and the like can be displayed, and confirmation of information and start of application can be smoothly performed.

As described above, the electronic devices can be obtained using the light-emitting device which is one embodiment of the present invention. Note that the light-emitting device can be used for electronic devices in a variety of fields without being limited to the electronic devices described in this embodiment.

Note that the structure described in this embodiment can be combined as appropriate with any of the structures described in other embodiments.

### Embodiment 6

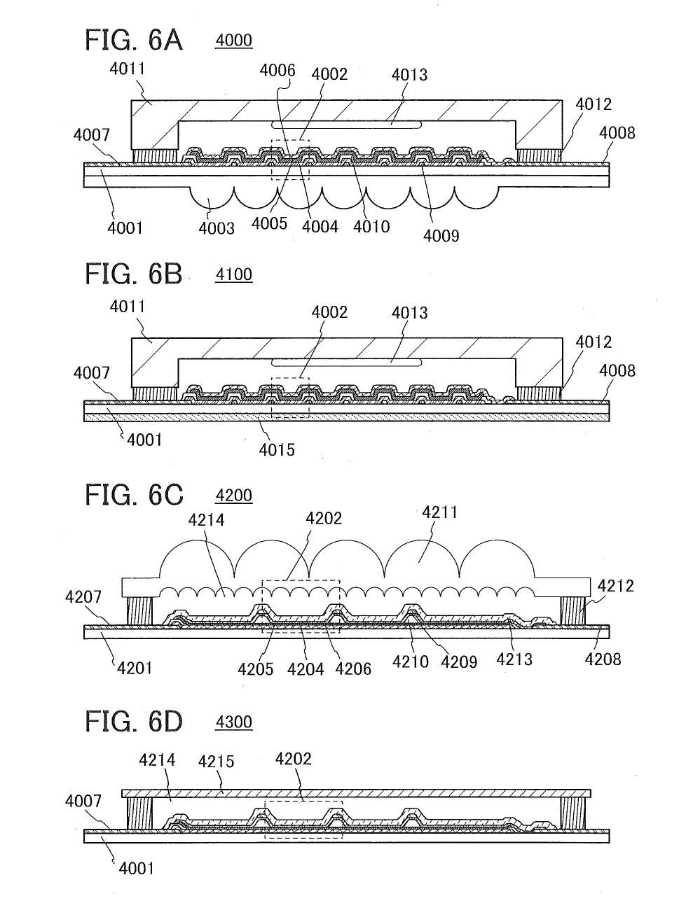

In this embodiment, a structure of a lighting device fabricated using the light-emitting element of one embodiment of the present invention is described with reference to FIGS. 6A to 6D.

FIGS. 6A to 6D are examples of cross-sectional views of lighting devices. FIGS. 6A and 6B illustrate bottom-emission lighting devices in which light is extracted from the substrate side, and FIGS. 6C and 6D illustrate top-emission lighting devices in which light is extracted from the sealing substrate side.

A lighting device 4000 illustrated in FIG. 6A includes a light-emitting element 4002 over a substrate 4001. In addition, the lighting device 4000 includes a substrate 4003 with unevenness on the outside of the substrate 4001. The light-emitting element 4002 includes a first electrode 4004, an EL layer 4005, and a second electrode 4006.

The first electrode 4004 is electrically connected to an electrode 4007, and the second electrode 4006 is electrically connected to an electrode 4008. In addition, an auxiliary wiring 4009 electrically connected to the first electrode 4004 may be provided. Note that an insulating layer 4010 is formed over the auxiliary wiring 4009.

The substrate 4001 and a sealing substrate 4011 are bonded to each other by a sealant 4012. A desiccant 4013 is preferably provided between the sealing substrate 4011 and the light-emitting element 4002. The substrate 4003 has the unevenness illustrated in FIG. 6A, whereby the extraction efficiency of light emitted from the light-emitting element 4002 can be increased.

Instead of the substrate 4003, a diffusion plate 4015 may be provided on the outside of a substrate 4001 as in a lighting device 4100 illustrated in FIG. 6B.

A lighting device 4200 illustrated in FIG. 6C includes a light-emitting element 4202 over a substrate 4201. The light-emitting element 4202 includes a first electrode 4204, an EL layer 4205, and a second electrode 4206.

The first electrode 4204 is electrically connected to an electrode 4207, and the second electrode 4206 is electrically connected to an electrode 4208. An auxiliary wiring 4209 electrically connected to the second electrode 4206 may be provided. An insulating layer 4210 may be provided under the auxiliary wiring 4209.

The substrate 4201 and a sealing substrate 4211 with unevenness are bonded to each other by a sealant 4212. A barrier film 4213 and a planarization film 4214 may be provided between the sealing substrate 4211 and the light-emitting element 4202. The sealing substrate 4211 has the unevenness illustrated in FIG. 6C, whereby the extraction efficiency of light emitted from the light-emitting element 4202 can be increased.

Instead of the sealing substrate 4211, a diffusion plate 4215 may be provided over the light-emitting element 4202 as in a lighting device 4300 illustrated in FIG. 6D.

Note that the EL layers 4005 and 4205 in this embodiment can include the organic compound of one embodiment of the present invention. In that case, a lighting device with low power consumption can be provided.

Note that the structure described in this embodiment can be combined as appropriate with any of the structures described in other embodiments.

### Embodiment 7

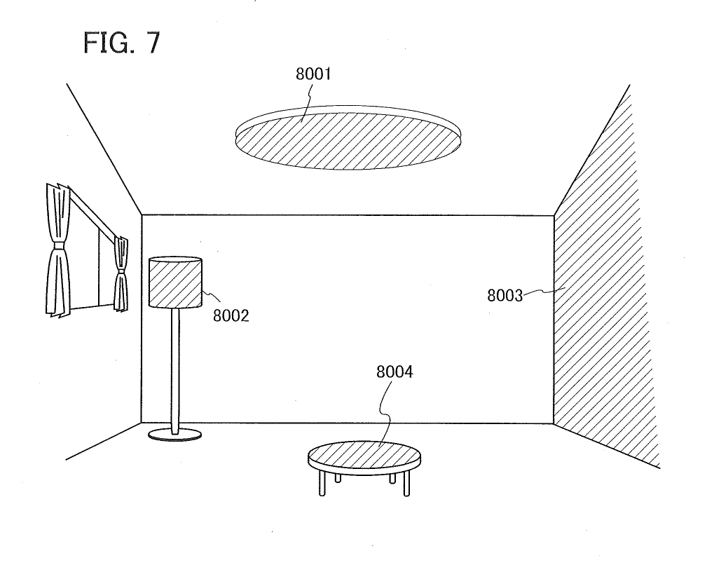

In this embodiment, examples of a lighting device that is an application of the light-emitting device in Embodiment 4 are described with reference to FIG. 7.

FIG. 7 illustrates an example in which the light-emitting device is used as an indoor lighting device 8001. Since the light-emitting device can have a large area, it can be used for a lighting device having a large area. In addition, with the use of a housing with a curved surface, a lighting device 8002 in which a light-emitting region has a curved surface can also be obtained. A light-emitting element included in the light-emitting device described in this embodiment is in a thin film form, which allows the housing to be designed more freely. Thus, the lighting device can be elaborately designed in a variety of ways. In addition, a wall of the room may be provided with a large-sized lighting device 8003.

When the light-emitting device is used for a surface of a table, a lighting device 8004 that has a function as a table can be obtained. When the light-emitting device is used as part of other furniture, a lighting device that functions as the furniture can be obtained.

As described above, a variety of lighting devices that include the light-emitting device can be obtained. Note that these lighting devices are also embodiments of the present invention.

Note that the structure described in this embodiment can be combined as appropriate with any of the structures described in other embodiments.

### Embodiment 8

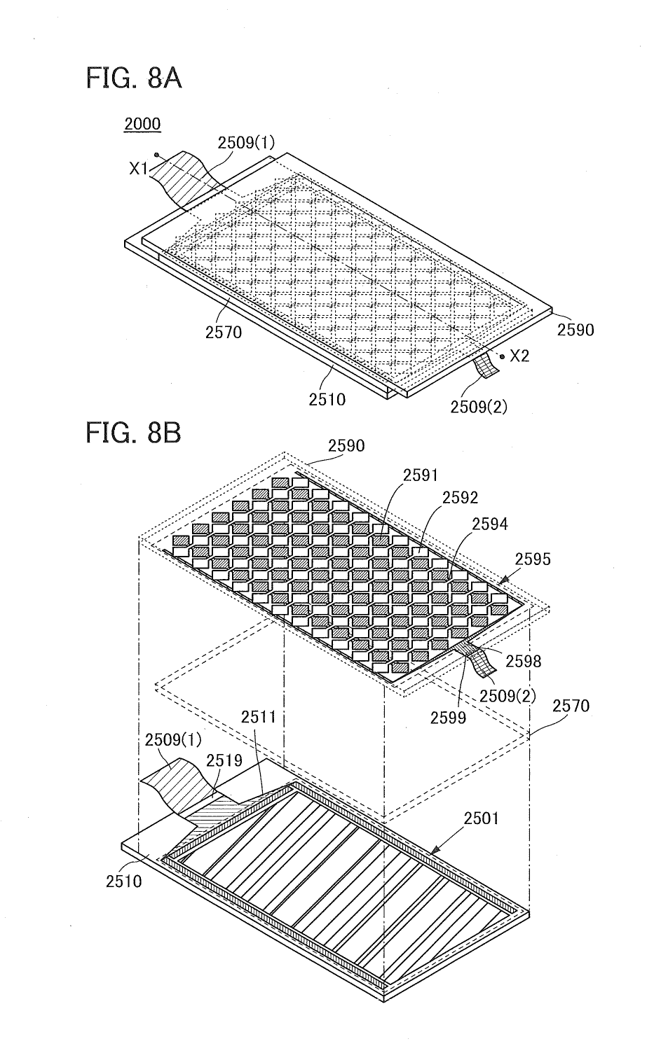

In this embodiment, touch panels including a light-emitting element of one embodiment of the present invention or a light-emitting device of one embodiment of the present invention are described with reference to FIGS. 8A and 8B, FIGS. 9A and 9B, FIGS. 10A and 10B, FIGS. 11A and 11B, and FIG. 12.

FIGS. 8A and 8B are perspective views of a touch panel 2000. Note that FIGS. 8A and 8B illustrate typical components of the touch panel 2000 for simplicity.

The touch panel 2000 includes a display panel 2501 and a touch sensor 2595 (see FIG. 8B). Furthermore, the touch panel 2000 includes a substrate 2510, a substrate 2570, and a substrate 2590.

The display panel 2501 includes a plurality of pixels over the substrate 2510, and a plurality of wirings 2511 through which signals are supplied to the pixels. The plurality of wirings 2511 are led to a peripheral portion of the substrate 2510, and part of the plurality of wirings 2511 forms a terminal 2519. The terminal 2519 is electrically connected to an FPC 2509(1).

The substrate 2590 includes the touch sensor 2595 and a plurality of wirings 2598 electrically connected to the touch sensor 2595. The plurality of wirings 2598 are led to a peripheral portion of the substrate 2590, and part of the plurality of wirings 2598 forms a terminal 2599. The terminal 2599 is electrically connected to an FPC 2509(2). Note that in FIG. 8B, electrodes, wirings, and the like of the touch sensor 2595 provided on the back side of the substrate 2590 (the side facing the substrate 2510) are indicated by solid lines for clarity.

As the touch sensor 2595, a capacitive touch sensor can be used, for example. Examples of the capacitive touch sensor are a surface capacitive touch sensor and a projected capacitive touch sensor.

Examples of the projected capacitive touch sensor are a self capacitive touch sensor and a mutual capacitive touch sensor, which differ mainly in the driving method. The use of a mutual capacitive touch sensor is preferable because multiple points can be sensed simultaneously.

First, an example of using a projected capacitive touch sensor is described below with reference to FIG. 8B. Note that for a projected capacitive touch sensor, a variety of sensors that can sense the closeness or the contact of a sensing target such as a finger can be used.

The projected capacitive touch sensor 2595 includes electrodes 2591 and electrodes 2592. The electrodes 2591 are electrically connected to any of the plurality of wirings 2598, and the electrodes 2592 are electrically connected to any of the other wirings 2598. The electrodes 2592 each have a shape of a plurality of quadrangles arranged in one direction with one corner of a quadrangle connected to one corner of another quadrangle with a wiring 2594 in one direction as illustrated in FIGS. 8A and 8B. In the same manner, the electrodes 2591 each have a shape of a plurality of quadrangles arranged with one corner of a quadrangle connected to one corner of another quadrangle; however, the direction in which the electrodes 2591 are connected is a direction crossing the direction in which the electrodes 2592 are connected. Note that the direction in which the electrodes 2591 are connected and the direction in which the electrodes 2592 are connected are not necessarily perpendicular to each other, and the electrodes 2591 may be arranged to intersect with the electrodes 2592 at an angle greater than 0° and less than 90°.

The intersecting area of the wiring 2594 and one of the electrodes 2592 is preferably as small as possible. Such a structure allows a reduction in the area of a region where the electrodes are not provided, reducing unevenness in transmittance. As a result, unevenness in the luminance of light from the touch sensor 2595 can be reduced.

Note that the shapes of the electrodes 2591 and the electrodes 2592 are not limited to the above-mentioned shapes and can be any of a variety of shapes. For example, the plurality of electrodes 2591 may be provided so that space between the electrodes 2591 are reduced as much as possible, and the plurality of electrodes 2592 may be provided with an insulating layer sandwiched between the electrodes 2591 and the electrodes 2592. In that case, between two adjacent electrodes 2592, a dummy electrode which is electrically insulated from these electrodes is preferably provided, whereby the area of a region having a different transmittance can be reduced.

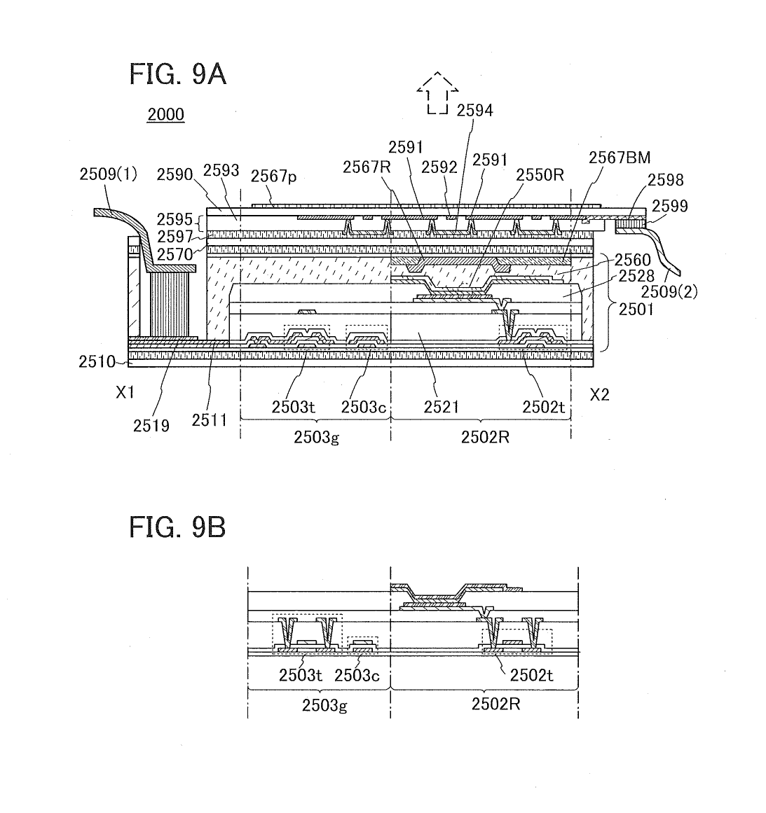

Next, the touch panel 2000 is described in detail with reference to FIGS. 9A and 9B. FIGS. 9A and 9B are cross-sectional views taken along the dashed-dotted line X1-X2 in FIG. 8A.

The touch panel 2000 includes the touch sensor 2595 and the display panel 2501.

The touch sensor 2595 includes the electrodes 2591 and the electrodes 2592 that are provided in a staggered arrangement and in contact with the substrate 2590, an insulating layer 2593 covering the electrodes 2591 and the electrodes 2592, and the wiring 2594 that electrically connects the adjacent electrodes 2591 to each other. Between the adjacent electrodes 2591, the electrode 2592 is provided.

The electrodes 2591 and the electrodes 2592 can be formed using a light-transmitting conductive material. As a light-transmitting conductive material, a conductive oxide such as indium oxide, indium tin oxide, indium zinc oxide, zinc oxide, or zinc oxide to which gallium is added can be used. A graphene compound may be used as well. When a graphene compound is used, it can be formed, for example, by reducing a graphene oxide film. As a reducing method, a method with application of heat, a method with laser irradiation, or the like can be employed.

For example, the electrodes 2591 and the electrodes 2592 can be formed by depositing a light-transmitting conductive material on the substrate 2590 by a sputtering method and then removing an unneeded portion by any of various patterning techniques such as photolithography.

Examples of a material for the insulating layer 2593 are a resin such as acrylic or epoxy resin, a resin having a siloxane bond, and an inorganic insulating material such as silicon oxide, silicon oxynitride, or aluminum oxide.

The adjacent electrodes 2591 are electrically connected to each other with a wiring 2594 formed in part of the insulating layer 2593. Note that a material for the wiring 2594 preferably has higher conductivity than materials for the electrode 2591 and the electrode 2592 to reduce electrical resistance.

One wiring 2598 is electrically connected to any of the electrodes 2591 and 2592. Part of the wiring 2598 serves as a terminal. For the wiring 2598, a metal material such as aluminum, gold, platinum, silver, nickel, titanium, tungsten, chromium, molybdenum, iron, cobalt, copper, or palladium or an alloy material containing any of these metal materials can be used.

Through the terminal 2599, the wiring 2598 and the FPC 2509(2) are electrically connected to each other. The terminal 2599 can be formed using any of various kinds of anisotropic conductive films (ACF), anisotropic conductive pastes (ACP), and the like.

An adhesive layer 2597 is provided in contact with the wiring 2594. That is, the touch sensor 2595 is attached to the display panel 2501 so that they overlap with each other with the adhesive layer 2597 provided therebetween. Note that the substrate 2570 as shown in FIG. 9A may be provided over the surface of the display panel 2501 that is adjacent to the adhesive layer 2597; however, the substrate 2570 is not always needed.

The adhesive layer 2597 has a light-transmitting property. For example, a thermosetting resin or an ultraviolet curable resin can be used; specifically, a resin such as an acrylic-based resin, a urethane-based resin, an epoxy-based resin, or a siloxane-based resin can be used.

The display panel 2501 in FIG. 9A includes, between the substrate 2510 and the substrate 2570, a plurality of pixels arranged in a matrix and a driver circuit. Each pixel includes a light-emitting element and a pixel circuit driving the light-emitting element.

In FIG. 9A, a pixel 2502R is shown as an example of the pixel of the display panel 2501, and a scan line driver circuit 2503g is shown as an example of the driver circuit.

The pixel 2502R includes Light-emitting Element 2550R and a transistor 2502t that can supply electric power to Light-emitting Element 2550R.

The transistor 2502t is covered with the insulating layer 2521. The insulating layer 2521 covers unevenness caused by the transistor and the like that have been already formed to provide a flat surface. The insulating layer 2521 may serve also as a layer for preventing diffusion of impurities. That is preferable because a reduction in the reliability of the transistor or the like due to diffusion of impurities can be prevented.

Light-emitting Element 2550R is electrically connected to the transistor 2502t through a wiring. It is one electrode of Light-emitting Element 2550R that is directly connected to the wiring. An end portion of the one electrode of Light-emitting Element 2550R is covered with an insulator 2528.

Light-emitting Element 2550R includes an EL layer between a pair of electrodes. A coloring layer 2567R is provided to overlap with Light-emitting Element 2550R, and part of light emitted from Light-emitting Element 2550R is transmitted through the coloring layer 2567R and extracted in the direction indicated by an arrow in the drawing. A light-blocking layer 2567BM is provided at an end portion of the coloring layer, and a sealing layer 2560 is provided between Light-emitting Element 2550R and the coloring layer 2567R.

Note that when the sealing layer 2560 is provided on the side from which light from Light-emitting Element 2550R is extracted, the sealing layer 2560 preferably has a light-transmitting property. The sealing layer 2560 preferably has a higher refractive index than the air.

A scan line driver circuit 2503g includes a transistor 2503t and a capacitor 2503c. Note that the driver circuit and the pixel circuits can be formed in the same process over the same substrate. Thus, similarly to the transistor 2502t in the pixel circuit, the transistor 2503t in the driver circuit (scan line driver circuit 2503g) is also covered with the insulating layer 2521.

The wirings 2511 through which a signal can be supplied to the transistor 2503t are provided. The terminal 2519 is provided in contact with the wiring 2511. The terminal 2519 is electrically connected to the FPC 2509(1), and the FPC 2509(1) has a function of supplying signals such as a pixel signal and a synchronization signal. Note that a printed wiring board (PWB) may be attached to the FPC 2509(1).

Although the case where the display panel 2501 shown in FIG. 9A includes a bottom-gate transistor is described, the structure of the transistor is not limited thereto, and any of transistors with various structures can be used. In each of the transistor 2502t and the transistor 2503t illustrated in FIG. 9A, a semiconductor layer including an oxide semiconductor can be used for a channel region. Alternatively, a semiconductor layer containing amorphous silicon or a semiconductor layer containing polycrystalline silicon that is obtained by crystallization process such as laser annealing can be used for a channel region.

FIG. 9B illustrates the structure of the display panel 2501 that includes a top-gate transistor instead of the bottom-gate transistor illustrated in FIG. 9A. The kind of the semiconductor layer that can be used for the channel region does not depend on the structure of the transistor.

In the touch panel 2000 shown in FIG. 9A, an anti-reflection layer 2567p overlapping with at least the pixel is preferably provided on a surface of the touch panel on the side from which light from the pixel is extracted, as shown in FIG. 9A. As the anti-reflection layer 2567p, a circular polarizing plate or the like can be used.

For the substrate 2510, the substrate 2570, and the substrate 2590 in FIG. 9A, for example, a flexible material having a vapor permeability of 1×10⁻⁵ g/(m²·day) or lower, preferably 1×10⁻⁶ g/(m²·day) or lower can be favorably used. Alternatively, it is preferable to use the materials that make these substrates have substantially the same coefficient of thermal expansion. For example, the coefficients of linear expansion of the materials are 1×10⁻³/K or lower, preferably 5×10⁻⁵/K or lower, and further preferably 1×10⁻⁵/K or lower.

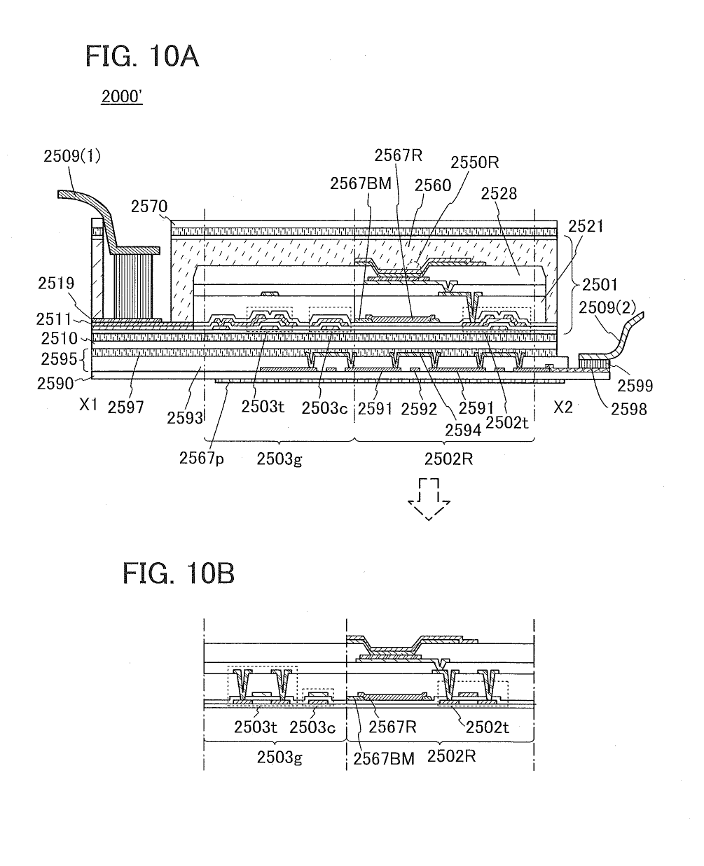

Next, a touch panel 2000′ having a structure different from that of the touch panel 2000 shown in FIGS. 9A and 9B is described with reference to FIGS. 10A and 10B. Note that the touch panel 2000′ can be used for an application similar to that of the touch panel 2000.

FIGS. 10A and 10B are cross-sectional views of the touch panel 2000′. In the touch panel 2000′ illustrated in FIGS. 10A and 10B, the position of the touch sensor 2595 relative to the display panel 2501 is different from that in the touch panel 2000 illustrated in FIGS. 9A and 9B. Only different structures are described below, and the above description of the touch panel 2000 can be referred to for the other similar structures.

The coloring layer 2567R overlaps with Light-emitting Element 2550R. Light from Light-emitting Element 2550R illustrated in FIG. 10A is emitted to the side where the transistor 2502t is provided. That is, (part of) light emitted from Light-emitting Element 2550R passes through the coloring layer 2567R and is extracted in the direction indicated by an arrow in FIG. 10A. Note that the light-blocking layer 2567BM is provided at an end portion of the coloring layer 2567R.

The touch sensor 2595 is provided on the side of the display panel 2501 that is closer to the transistor 2502t than to Light-emitting Element 2550R (see FIG. 10A).

The adhesive layer 2597 is in contact with the substrate 2510 of the display panel 2501 and attaches the display panel 2501 and the touch sensor 2595 to each other in the structure shown in FIG. 10A. The substrate 2510 is not necessarily provided between the display panel 2501 and the touch sensor 2595 that are attached to each other by the adhesive layer 2597.

As in the touch panel 2000, transistors with a variety of structures can be used for the display panel 2501 in the touch panel 2000′. Although a bottom-gate transistor is used in FIG. 10A, a top-gate transistor may be applied as shown in FIG. 10B.

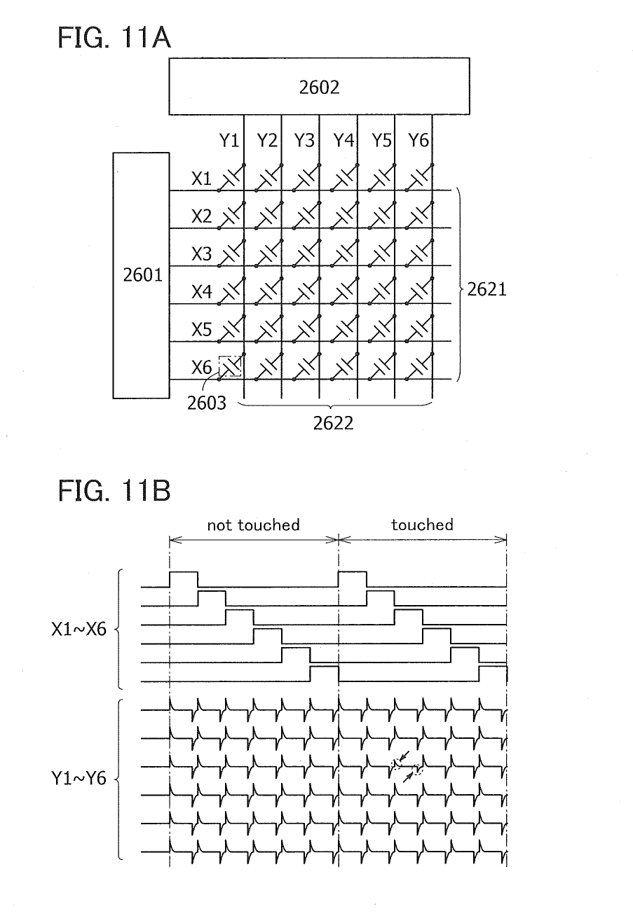

Then, an example of a driving method of the touch panel is described with reference to FIGS. 11A and 11B.

FIG. 11A is a block diagram illustrating the structure of a mutual capacitive touch sensor. FIG. 11A illustrates a pulse voltage output circuit 2601 and a current sensing circuit 2602. Note that in the example of FIG. 11A, six wirings X1-X6 represent electrodes 2621 to which a pulse voltage is supplied, and six wirings Y1-Y6 represent electrodes 2622 that sense a change in current. FIG. 11A also illustrates a capacitor 2603 that is formed in a region where the electrodes 2621 and 2622 overlap with each other. Note that functional replacement between the electrodes 2621 and 2622 is possible.

The pulse voltage output circuit 2601 is a circuit for sequentially applying a pulse voltage to the wirings X1 to X6. By application of a pulse voltage to the wirings X1 to X6, an electric field is generated between the electrodes 2621 and 2622 of the capacitor 2603. When the electric field between the electrodes is shielded, for example, a change occurs in the capacitor 2603 (mutual capacitance). The approach or contact of a sensing target can be sensed by utilizing this change.

The current sensing circuit 2602 is a circuit for sensing changes in current flowing through the wirings Y1 to Y6 that are caused by the change in mutual capacitance in the capacitor 2603. No change in current value is sensed in the wirings Y1 to Y6 when there is no approach or contact of a sensing target, whereas a decrease in current value is sensed when mutual capacitance is decreased owing to the approach or contact of a sensing target. Note that an integrator circuit or the like is used for sensing of current.

FIG. 11B is a timing chart showing input and output waveforms in the mutual capacitive touch sensor illustrated in FIG. 11A. In FIG. 11B, sensing of a sensing target is performed in all the rows and columns in one frame period. FIG. 11B shows a period when a sensing target is not sensed (not touched) and a period when a sensing target is sensed (touched). Sensed current values of the wirings Y1 to Y6 are shown as the waveforms of voltage values.

A pulse voltage is sequentially applied to the wirings X1 to X6, and the waveforms of the wirings Y1 to Y6 change in accordance with the pulse voltage. When there is no approach or contact of a sensing target, the waveforms of the wirings Y1 to Y6 change in accordance with changes in the voltages of the wirings X1 to X6. The current value is decreased at the point of approach or contact of a sensing target and accordingly the waveform of the voltage value changes. By sensing a change in mutual capacitance in this manner, the approach or contact of a sensing target can be sensed.

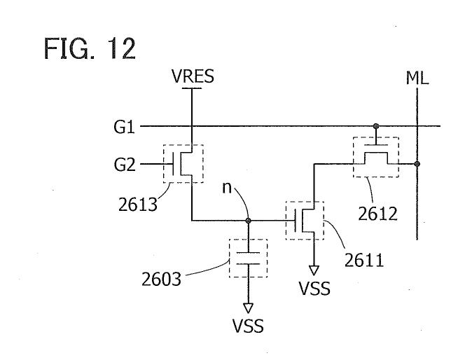

Although FIG. 11A illustrates a passive touch sensor in which only the capacitor 2603 is provided at the intersection of wirings as a touch sensor, an active touch sensor including a transistor and a capacitor may be used. FIG. 12 is a sensor circuit included in an active touch sensor.

The sensor circuit illustrated in FIG. 12 includes the capacitor 2603, a transistor 2611, a transistor 2612, and a transistor 2613.

A signal G2 is input to a gate of the transistor 2613. A voltage VRES is applied to one of a source and a drain of the transistor 2613, and one electrode of the capacitor 2603 and a gate of the transistor 2611 are electrically connected to the other of the source and the drain of the transistor 2613. One of a source and a drain of the transistor 2611 is electrically connected to one of a source and a drain of the transistor 2612, and a voltage VSS is applied to the other of the source and the drain of the transistor 2611. A signal G1 is input to a gate of the transistor 2612, and a wiring ML is electrically connected to the other of the source and the drain of the transistor 2612. The voltage VSS is applied to the other electrode of the capacitor 2603.

Next, the operation of the sensor circuit illustrated in FIG. 12 is described. First, a potential for turning on the transistor 2613 is supplied as the signal G2, and a potential with respect to the voltage VRES is thus applied to the node n connected to the gate of the transistor 2611. Then, a potential for turning off the transistor 2613 is applied as the signal G2, whereby the potential of the node n is maintained. Then, mutual capacitance of the capacitor 2603 changes owing to the approach or contact of a sensing target such as a finger, and accordingly the potential of the node n is changed from VRES.

In reading operation, a potential for turning on the transistor 2612 is supplied as the signal G1. A current flowing through the transistor 2611, that is, a current flowing through the wiring ML is changed in accordance with the potential of the node n. By sensing this current, the approach or contact of a sensing target can be sensed.

In each of the transistors 2611, 2612, and 2613, an oxide semiconductor layer is preferably used as a semiconductor layer in which a channel region is formed. In particular, such a transistor is preferably used as the transistor 2613 so that the potential of the node n can be held for a long time and the frequency of operation of resupplying VRES to the node n (refresh operation) can be reduced.

At least part of this embodiment can be implemented in combination with any of other embodiments described in this specification as appropriate.

### Example 1

### Synthesis Example 1

Example 1 describes a method for synthesizing 2-[3′-(dibenzodiophen-4-yl)(1,1′-biphenyl-3-yl)]dibenzo[f,h]quinazoline (abbreviation: 2mDBtBPDBqz), which is the organic compound of one embodiment of the present invention represented by Structural Formula (100) in Embodiment 1. The structure of 2mDBtBPDBqz is shown below.

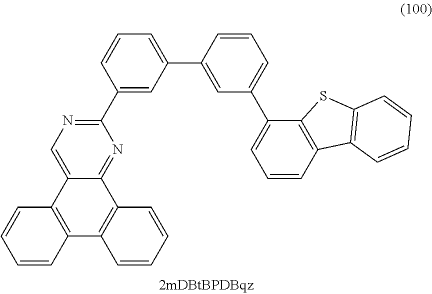 [SMILES: c1cc(-c2cccc(-c3cccc4c3sc3ccccc34)c2)cc(-c2ncc3c4ccccc4c4ccccc4c3n2)c1]

### Step 1: Synthesis of N-(phenanthren-9-yl)-3-chlorobenzamidine

Into an 80-mL flask were put 3.27 g (17 mmol) of 3-chlorobenzamidine hydrochloride, 5.2 g (17 mmol) of 9-iodophenanthren, 0.31 g (1.6 mmol) of copper iodide, 16 g (49 mmol) of cesium carbonate, 0.29 g (3.3 mmol) of N,N-dimethylethylenediamine, and 80 mL of dimethylformamide (DMF), and the air in the flask was replaced with nitrogen. The mixture in the flask was stirred at 100° C. for 21 hours. Then, 0.31 g (1.6 mmol) of copper iodide and 0.29 g (3.3 mmol) of N,N-dimethylethylenediamine were added, and the mixture was stirred at 110° C. for 8.5 hours. The resulting solution was subjected to suction filtration, and the obtained solid was washed with toluene. The obtained filtrate was washed with water, and the organic layer was washed with saturated saline. Magnesium sulfate was added to the organic layer for drying, and the resulting mixture was subjected to gravity filtration to give a filtrate. The filtrate was condensed to obtain an objective substance (a brown oily substance, at a yield of 50%). The synthesis scheme of Step 1 is shown in (a-1).

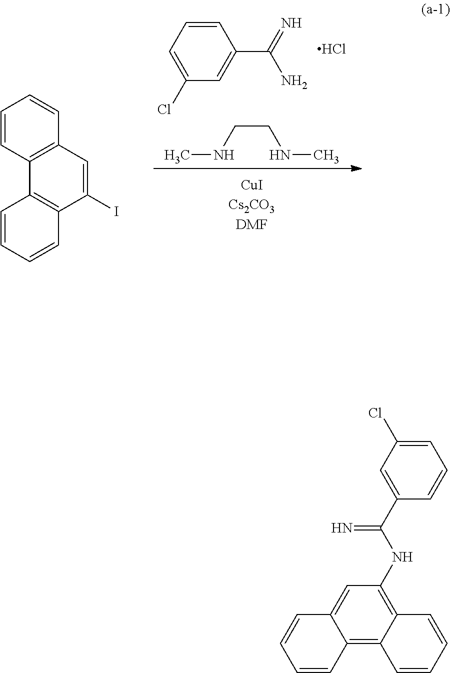

### Step 2: Synthesis of 2-(3-chlorophenyl)dibenzo[f,h]quinazoline

Next, 2.8 g (8.4 mmol) of N-(phenanthren-9-yl)-3-chlorobenzamidine and 50 mL of N,N′-dimethylformamide diethyl acetal were put into a 300 mL-flask, and the mixture was refluxed at 120° C. for one hour. After a predetermined time has passed, the resulting solution was subjected to suction filtration, and the obtained solid was washed with ethanol. The solid was recrystallized with toluene, and an objective substance was obtained (a pale red solid, at a yield of 77%). The synthesis scheme of Step 2 is shown in (a-2).

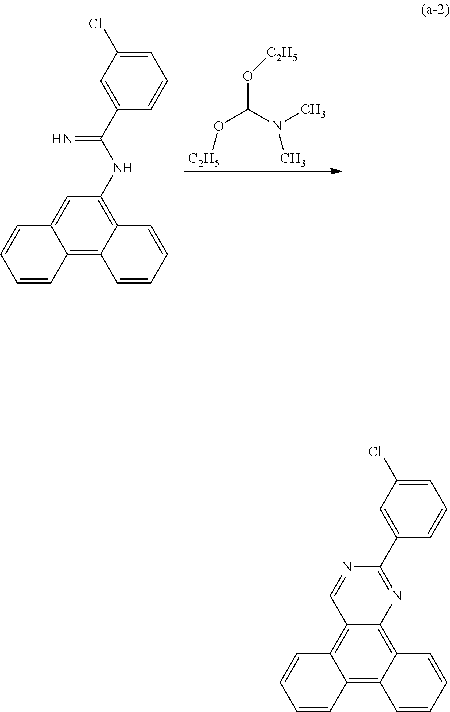

### Step 3: Synthesis of 2-[3′-(dibenzodiophen-4-yl)(1,1′-biphenyl-3-yl)]dibenzo[f,h]quinazoline (abbreviation: 2mDBtBPDBqz)

Next, 2.1 g (6.2 mmol) of 2-(3-chlorophenyfidibenzo[f,h]quinazoline, 2.1 g (6.8 mmol) of 4-dibenzothiophen-4-yl-phenylboronic acid, 44 mg (0.12 mmol) of di(1-adamantyl)-n-butylphosphine (cataCXium®), 3.9 g (19 mmol) of tripotassium phosphate, 41 mL of dioxane, and 1.4 g (19 mmol) of tert-butanol were put into a three-neck flask, and the air in the flask was replaced with nitrogen. To this mixture was added 14 mg (0.062 mmol) of palladium(II) acetate, and the mixture was stirred at 80° C. for four hours and at 100° C. for 19 hours. The resulting solution was subjected to suction filtration, and the obtained solid was washed with water and ethanol. The obtained solid was dissolved in toluene and subjected to suction filtration through a stack of Celite, alumina, and Celite. The obtained filtrate was concentrated to give a solid, and the solid was washed with boiling toluene to give an objective substance (a white solid, at a yield of 52%). The synthesis scheme of Step 3 is shown in (a-3).

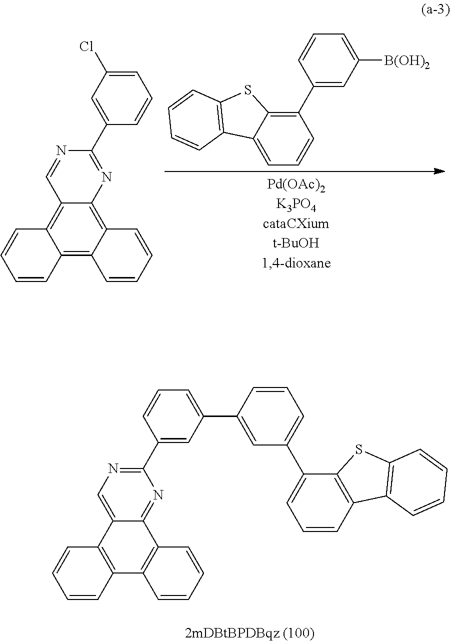

The obtained solid was purified by a train sublimation method. The purification by sublimation was carried out under a pressure of 2.5×10⁻² Pa and at a heating temperature of 290° C. After the purification by sublimation, a white solid which was an objective substance was obtained at a collection rate of 46%.

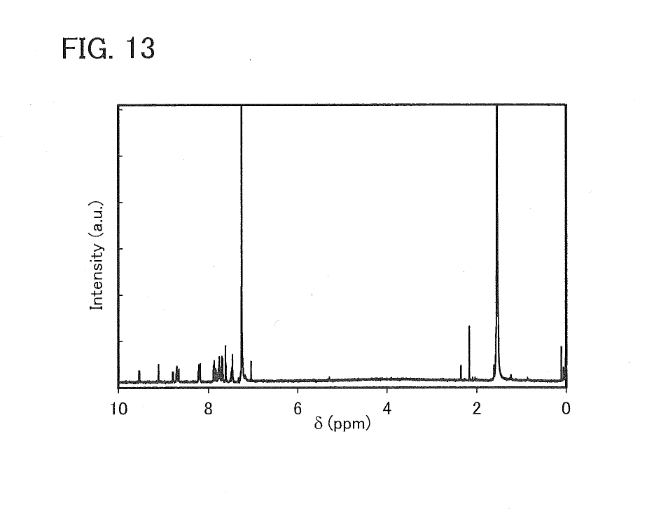

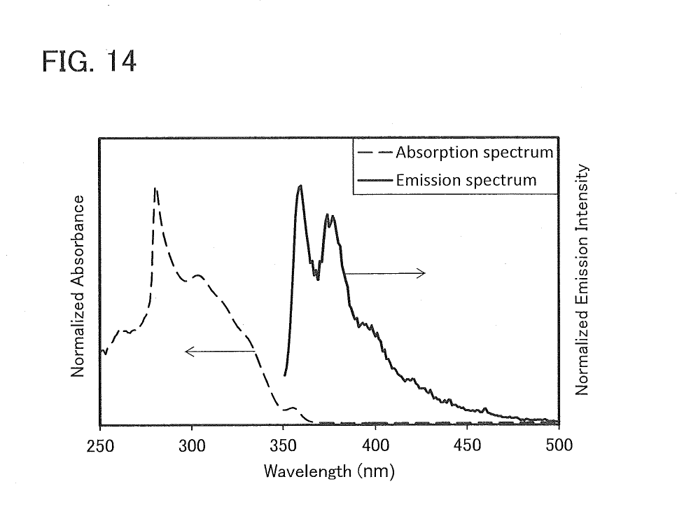

The result of analysis by nuclear magnetic resonance (¹H-NMR) spectroscopy of the white solid obtained in Step 3 is described below. The ¹H-NMR chart is shown in FIG. 13. The results revealed that 2mDBTBPDBqz, the organic compound of one embodiment of the present invention represented by Structural Formula (100), was obtained in Synthesis Example 1.

¹H-NMR. δ (CDCl₃): 7.47-7.50 (m, 2H), 7.61-7.63 (m, 2H), 7.68-7.89 (m, 10H), 8.18-8.23 (m, 3H), 8.67 (d, 1H), 8.70 (dd, 2H), 8.79 (d, 1H), 9.11 (s, 1H), 9.54 (d, 1H), 10.1 (s, 1H).

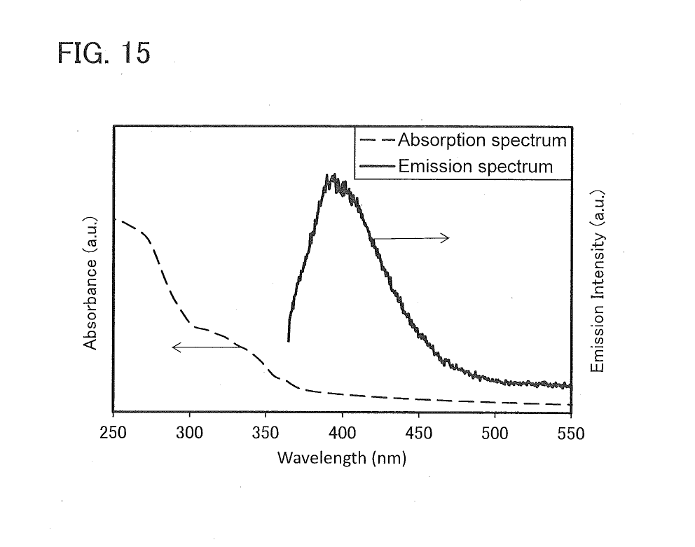

Next, the ultraviolet-visible absorption spectrum (hereinafter, simply referred to as “absorption spectrum”) and emission spectrum of 2mDBtBPDBqz in a toluene solution and in a solid thin film were measured. The solid thin film was formed over a quartz substrate by a vacuum evaporation method. The absorption spectrum was measured using an ultraviolet-visible light spectrophotometer (V550 type manufactured by JASCO Corporation). The emission spectrum was measured using a fluorescence spectrophotometer (FS920 manufactured by Hamamatsu Photonics K.K.). The results of the measurement of absorption spectrum and emission spectrum of the toluene solution are shown in FIG. 14. In FIG. 14, the horizontal axis represents wavelength and the vertical axes represent absorbance and emission intensity. The results of the measurement of absorption spectrum and emission spectrum of the solid thin film are shown in FIG. 15. In FIG. 15, the horizontal axis represents wavelength and the vertical axes represent absorbance and emission intensity.

FIG. 14 shows that 2mDBtBPDBqz in the toluene solution has absorption peaks at around 331 nm and 356 nm, and emission wavelength peaks at around 360 nm and 377 nm. FIG. 15 shows that 2mDBtBPDBqz in the solid thin film has absorption peaks at around 338 nm and 363 nm, and an emission wavelength peak at around 394 nm.

### Example 2

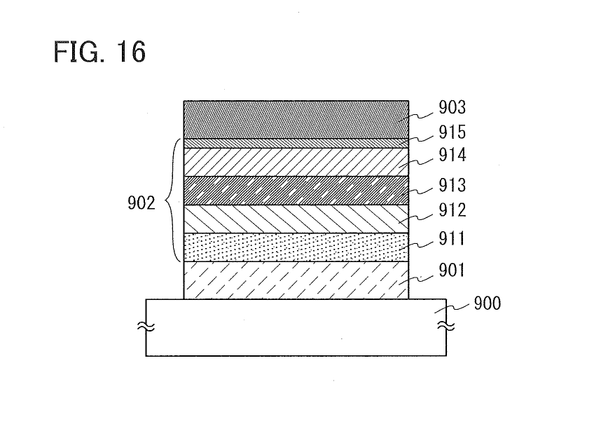

In Example 2, Light-emitting Element 1 in which 2mDBtBPDBqz (Structural Formula (100)), the organic compound of one embodiment of the present invention, was used for a light-emitting layer was fabricated, and the emission spectrum was measured. The fabrication of Light-emitting Element 1 is described with reference to FIG. 16. Chemical formulae of materials used in Example 2 are shown below.

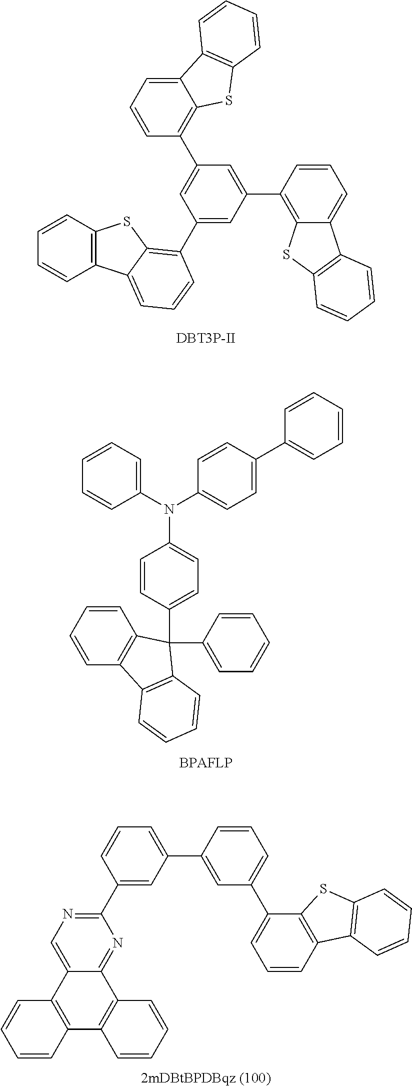

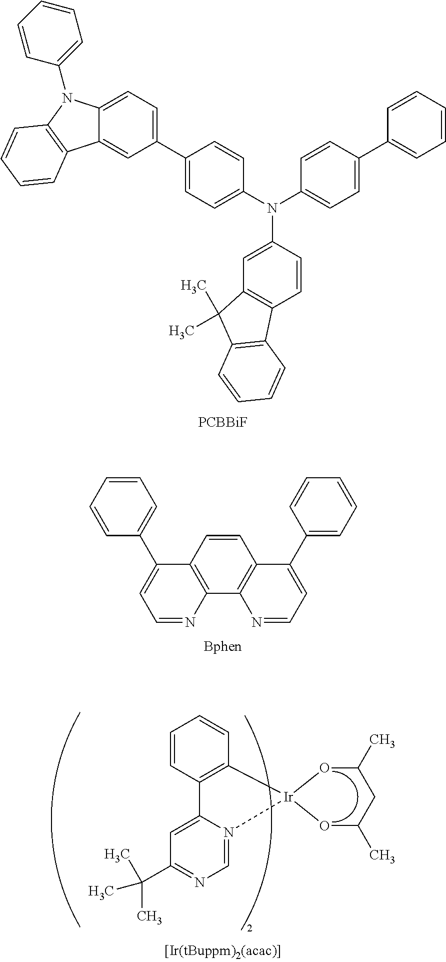

### <<Fabrication of Light-Emitting Element 1>>

First, indium tin oxide containing silicon oxide (ITO-1) was deposited over a glass substrate 900 by a sputtering method, whereby a first electrode 901 which functions as an anode was formed. The film thickness and the area of the first electrode were set to be 110 nm and 2 mm×2 mm, respectively.

Next, as pretreatment for fabricating Light-emitting Element 1 over the substrate 900, UV ozone treatment was performed for 370 seconds after washing of a surface of the substrate with water and baking performed at 200° C. for one hour.

After that, the substrate 900 was transferred into a vacuum evaporation apparatus where the pressure had been reduced to approximately 1×10⁻⁴ Pa, and was subjected to vacuum baking at 170° C. for 30 minutes in a heating chamber of the vacuum evaporation apparatus. Then, the substrate 900 was cooled down for approximately 30 minutes.

Next, the substrate 900 was fixed to a holder provided inside the vacuum evaporation apparatus such that the surface over which the first electrode 901 was formed faced downward. In this example, a case is described in which a hole-injection layer 911, a hole-transport layer 912, a light-emitting layer 913, an electron-transport layer 914, and an electron-injection layer 915 which are included in an EL layer 902 are sequentially formed by a vacuum evaporation method.

After reducing the pressure of the vacuum evaporation apparatus to 1×10⁻⁴ Pa, 1,3,5-tri(dibenzothiophen-4-yl)benzene (abbreviation: DBT3P-II) and molybdenum oxide were deposited by co-evaporation such that the mass ratio of DBT3P-II to molybdenum oxide becomes 4:2, whereby the hole-injection layer 911 was formed over the first electrode 901. The thickness thereof was set to be 20 nm. Note that a co-evaporation method is an evaporation method in which a plurality of different substances is concurrently vaporized from different evaporation sources.

Then, 4-phenyl-4′-(9-phenylfluoren-9-yl)triphenylamine (abbreviation: BPAFLP) was deposited by evaporation to a thickness of 20 nm, whereby the hole-transport layer 912 was formed.

Next, the light-emitting layer 913 was formed over the hole-transport layer 912. For the light-emitting layer 913, co-deposited by evaporation were 2-[3′-(dibenzodiophen-4-yl)(1,1′-biphenyl-3-yl)]dibenzo[f,h]quinazoline (abbreviation: 2mDBtBPDBqz), N-(1,1′-biphenyl-4-yl)-9,9-dimethyl-N-[4-(9-phenyl-9H-carbazol-3-yl)phenyl]-9H-fluor en-2-amine (abbreviation: PCBBiF), and [Ir(tBuppm)₂(acac)] with a mass ratio of 2mDBtBPDBqz to PCBBiF and [Ir(tBuppm)₂(acac)] being 0.7:0.3:0.05. The thickness was set to be 20 nm. In addition, 2mDBtBPDBqz, PCBBiF, and [Ir(tBuppm)₂(acac)] were co-deposited by evaporation with a mass ratio of 2mDBtBPDBqz to PCBBiF and [Ir(tBuppm)₂(acac)] being 0.8:0.2:0.05. The thickness was set to be 20 nm. Accordingly, the light-emitting layer 913 has a stacked-layer structure with a thickness of 40 nm.

Next, 2mDBtBPDBqz was deposited by evaporation to a thickness of 20 nm over the light-emitting layer 913 and then bathophenanthroline (abbreviation: Bphen) was deposited by evaporation to a thickness of 10 nm, whereby the electron-transport layer 914 was formed. Furthermore, lithium fluoride was deposited by evaporation to a thickness of 1 nm over the electron-transport layer 914, whereby the electron-injection layer 915 was formed.

Finally, aluminum was deposited by evaporation to a thickness of 200 nm over the electron-injection layer 915, whereby a second electrode 903 serving as a cathode is formed. In the above manner, Light-emitting Element 1 was obtained. Note that in all the above evaporation steps, evaporation was performed by a resistance-heating method.

Table 1 shows the element structure of Light-emitting Element 1 obtained in the above manner.

TABLE 1

| | | Hole- | Hole- | Light- | | | Electron- | |
|---|---|---|---|---|---|---|---|---|
| | First | injection | transport | emitting | | | injection | Second |
| | electrode | layer | layer | layer | Electron-transport layer | | layer | electrode |
| Light- | ITO-1 | DBT3P-II:MoOx | BPAFLP | * | 2mDBtBPDBqz | Bphen | LiF | Al |
| emitting | (110 nm) | (4:2 20 nm) | (20 mn) | | (20 nm) | (10 nm) | (1 nm) | (200 nm) |
| element 1 | | | | | | | | |

* 2mDBtBPDBqz:PCBBiF:[Ir(tBuppm)₂(acac)] (0.7:0.3:0.05 20 nm\0.8:0.2:0.05 20 nm)

The fabricated light-emitting element 1 was sealed in a glove box under a nitrogen atmosphere so as not to be exposed to the air (specifically, a sealant was applied to surround the element, and at the time of sealing, UV treatment was performed and heat treatment was performed at 80° C. for one hour).

### <<Operation Characteristics of Light-Emitting Element 1>>

Operation characteristics of the fabricated light-emitting element 1 were measured. The measurement was carried out at room temperature (in an atmosphere kept at 25° C.).

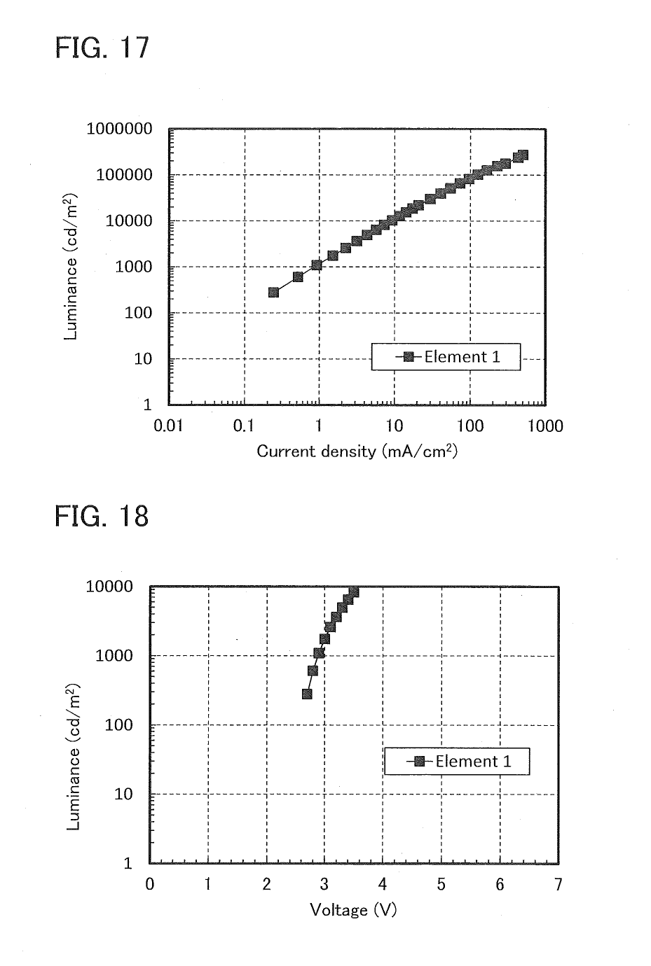

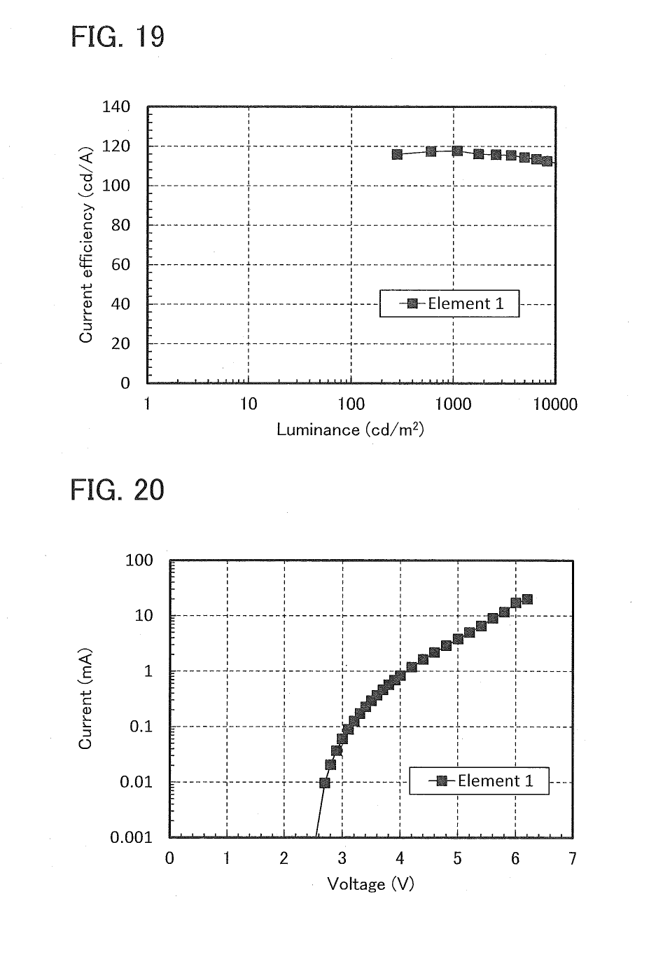

FIG. 17 shows current density-luminance characteristics of Light-emitting Element 1, FIG. 18 shows voltage-luminance characteristics of Light-emitting Element 1, FIG. 19 shows luminance-current efficiency characteristics of Light-emitting Element 1, and FIG. 20 shows voltage-current characteristics of Light-emitting Element 1.

These results reveal that Light-emitting Element 1 of one embodiment of the present invention has high efficiency. Table 2 shows main values of initial characteristics of Light-emitting Element 1 at a luminance of approximately 1000 cd/m².

TABLE 2

| | | | Current | | | Current | Power | External |
|---|---|---|---|---|---|---|---|---|
| | Voltage | Current | density | Chromaticity | Luminance | efficiency | efficiency | quantum |
| | (V) | (mA) | (mA/cm²) | (x, y) | (cd/m²) | (cd/A) | (lm/W) | efficiency (%) |
| Light- | 2.9 | 0.037 | 0.9 | (0.41, 0.58) | 1100 | 120 | 130 | 31 |
| emitting | | | | | | | | |
| element 1 | | | | | | | | |

The above results show that Light-emitting Element 1 fabricated in Example 2 has high current efficiency and high external quantum efficiency.

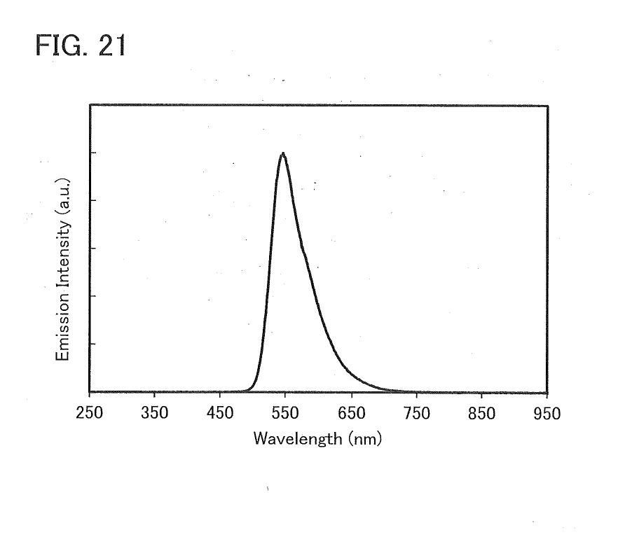

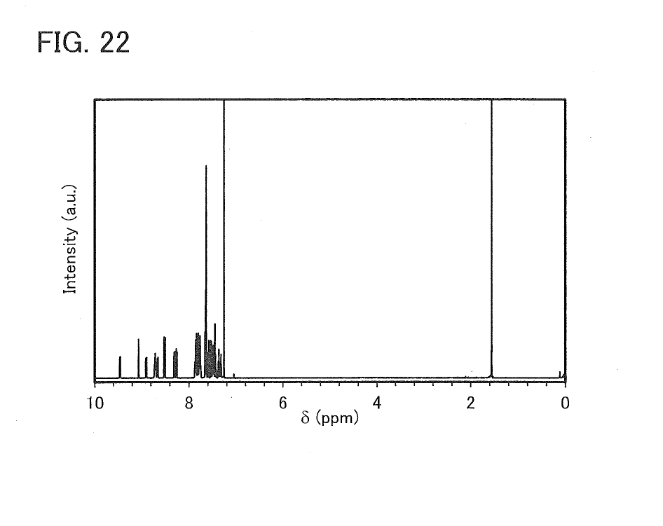

FIG. 21 shows an emission spectrum of Light-emitting Element 1 to which current was applied at a current density of 25 mA/cm². As shown in FIG. 21, the emission spectrum of Light-emitting Element 1 has a peak at around 541 nm, which indicates that the peak is derived from emission of an organometallic complex [Ir(tBuppm)₂(acac)] contained in the light-emitting layer 913. Note that 2mDBtBPDBqz used for the light-emitting layer and the electron-transport layer in Example 2 is the organic compound of one embodiment of the present invention which has a structure in which the 2-position of a dibenzoquinazoline ring is bonded to a skeleton with a hole-transport property via a 1,3-phenylene group, and is capable of raising the singlet level (S1) and the triplet level (T1) and widening the energy gap. Accordingly, as compared with a structure in which the 2-position of a dibenzoquinazoline ring is bonded to a skeleton with a hole-transport property via a 1,4-phenylene group, carrier balance can be easily adjusted and carrier passage can be prevented. Thus, the improvement in emission efficiency of Light-emitting Element 1 of Example 2 is attributable to the use of 2mDBtBPDBqz.

### Example 3

### Synthesis Example 2

Example 3 describes a method for synthesizing 2-[3-(9′-phenyl-3,3′-bi-9H-carbazol-9-yl)phenyl]dibenzo[f,h]quinazoline (abbreviation: 2mPCCzPDBqz), which is the organic compound of one embodiment of the present invention represented by Structural Formula (123) in Embodiment 1. The structure of 2mPCCzPDBqz is shown below.

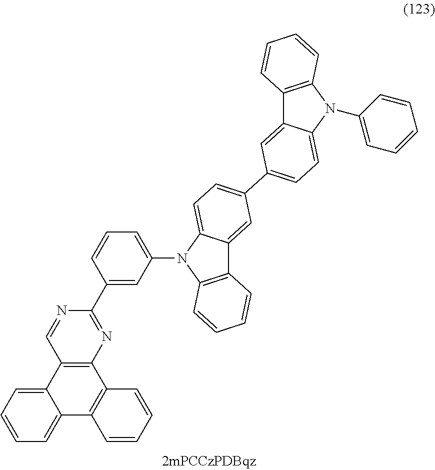 [SMILES: c1ccc(-n2c3ccccc3c3cc(-c4ccc5c(c4)c4ccccc4n5-c4cccc(-c5ncc6c7ccccc7c7ccccc7c6n5)c4)ccc32)cc1]

Into a three-neck flask were put 2.5 g (7.3 mmol) of 2-(3-chlorophenyl)dibenzo[f,h]quinazoline, 3.0 g (7.3 mmol) of 9-phenyl-3,3′-bi-9H-carbazole, 2.1 g (22 mmol) of sodium tert-butoxide, 120 mg (0.30 mmol) of 2-dicyclohexylphosphino-2′,6′-dimethoxybiphenyl (S-Phos), and 37 mL of mesitylene, and the air in the flask was replaced with nitrogen. To the mixture, 84 mg (0.146 mmol) of bis(dibenzylideneacetone)palladium(0) was added, and the resulting mixture was stirred at 130° C. for 21 hours.

The resulting solution was subjected to suction filtration, and the obtained solid was washed with water and ethanol. The solid was dissolved in toluene and subjected to suction filtration through a stack of Celite, alumina, and Celite. The obtained filtrate was concentrated to give a solid, and the solid was recrystallized with toluene to give an objective substance (a white solid, at a yield of 51%). The synthesis scheme of this synthesis is shown in (b-1).

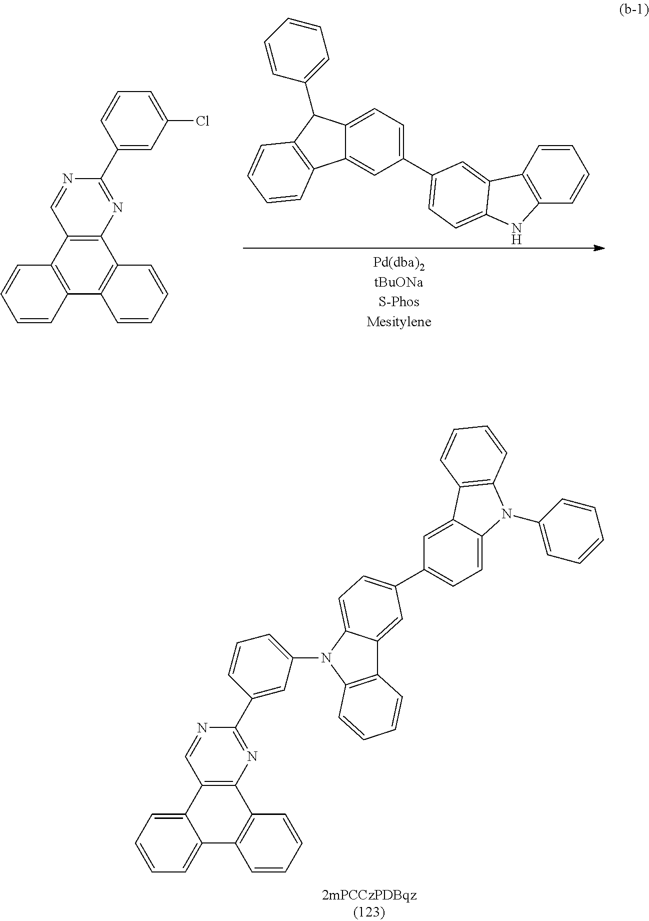 [SMILES: Clc1cccc(-c2ncc3c4ccccc4c4ccccc4c3n2)c1 ; c1ccc(C2c3ccccc3-c3cc(-c4ccc5[nH]c6ccccc6c5c4)ccc32)cc1 ; c1ccc(-n2c3ccccc3c3cc(-c4ccc5c(c4)c4ccccc4n5-c4cccc(-c5ncc6c7ccccc7c7ccccc7c6n5)c4)ccc32)cc1]

The obtained solid was purified by a train sublimation method. The purification by sublimation was carried out under a pressure of 3.4 Pa, with a flow rate of argon gas of 15 mL/min, and at a heating temperature of 385° C. After the purification by sublimation, a yellow solid which was an objective substance was obtained at a collection rate of 74%.

The result of analysis by nuclear magnetic resonance (¹H-NMR) spectroscopy of the yellow solid obtained in the above synthesis is described below. The ¹H-NMR chart is shown in FIG. 22. The result revealed that 2mPCCzPDBqz, the organic compound of one embodiment of the present invention represented by Structural Formula (123), was obtained in Synthesis Example 2.

¹H-NMR. δ (CDCl₃): 7.33 (t, 1H), 7.37 (t, 1H), 7.42-7.51 (m, 4H), 7.54 (d, 1H), 7.59 (d, 1H), 7.63-7.67 (m, 51H), 7.75-7.88 (m, 8H), 8.28 (dd, 2H), 8.50 (dd, 2H), 8.68 (d, 1H), 8.70 (t, 2H), 8.90 (d, 1H), 9.06 (s, 1H), 9.46 (d, 1H), 10.1 (s, 1H).

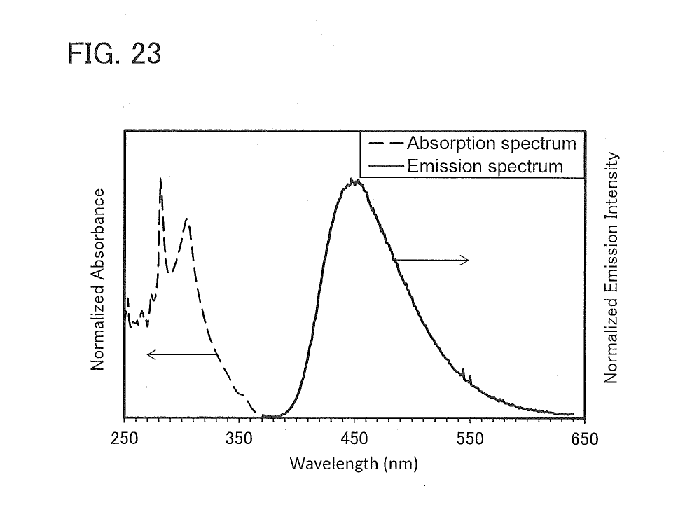

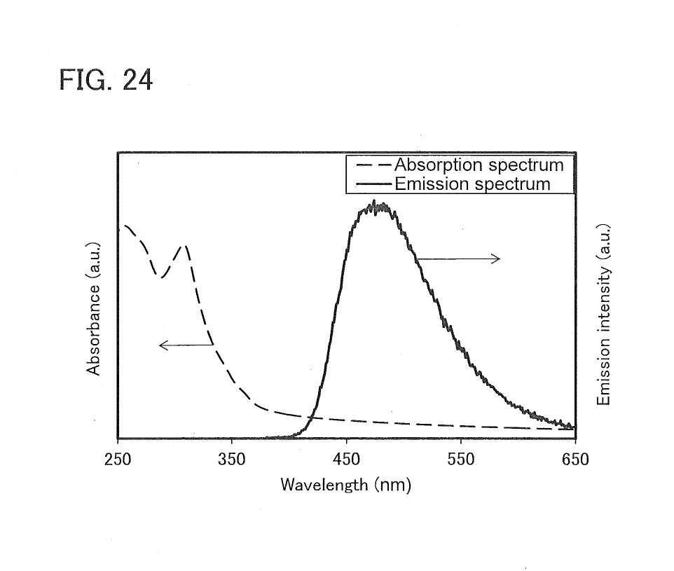

Next, the ultraviolet-visible absorption spectrum (hereinafter, simply referred to as “absorption spectrum”) and emission spectrum of 2mPCCzPDBqz in a toluene solution and in a solid thin film were measured. The solid thin film was formed over a quartz substrate by a vacuum evaporation method. The absorption spectrum was measured using an ultraviolet-visible light spectrophotometer (V550 type manufactured by JASCO Corporation). The emission spectrum was measured using a fluorescence spectrophotometer (FS920 manufactured by Hamamatsu Photonics K.K.). The results of the measurement of absorption spectrum and emission spectrum of the toluene solution are shown in FIG. 23. In FIG. 23, the horizontal axis represents wavelength and the vertical axes represent absorbance and emission intensity. The results of the measurement of absorption spectrum and emission spectrum of the solid thin film are shown in FIG. 24. In FIG. 24, the horizontal axis represents wavelength and the vertical axes represent absorbance and emission intensity.

FIG. 23 shows that 2mPCCzPDBqz in the toluene solution has absorption peaks at around 281 nm and 304 nm, and an emission wavelength peak at around 447 nm (excitation at 304 nm). FIG. 24 shows that 2mPCCzPDBqz in the solid thin film has absorption peaks at around 346 nm and 362 nm, and an emission wavelength peak at around 480 nm.

### Example 4

In Example 4, Light-emitting Element 2 in which 2mPCCzPDBqz (Structural Formula (123)), the organic compound of one embodiment of the present invention, was used for a light-emitting layer was fabricated, and the emission spectrum was measured. Note that description of details of Light-emitting Element 2 fabrication which are similar to those of Light-emitting Element 1 fabrication described in Example 2 is omitted. Chemical formulae of materials used in this example are shown below.

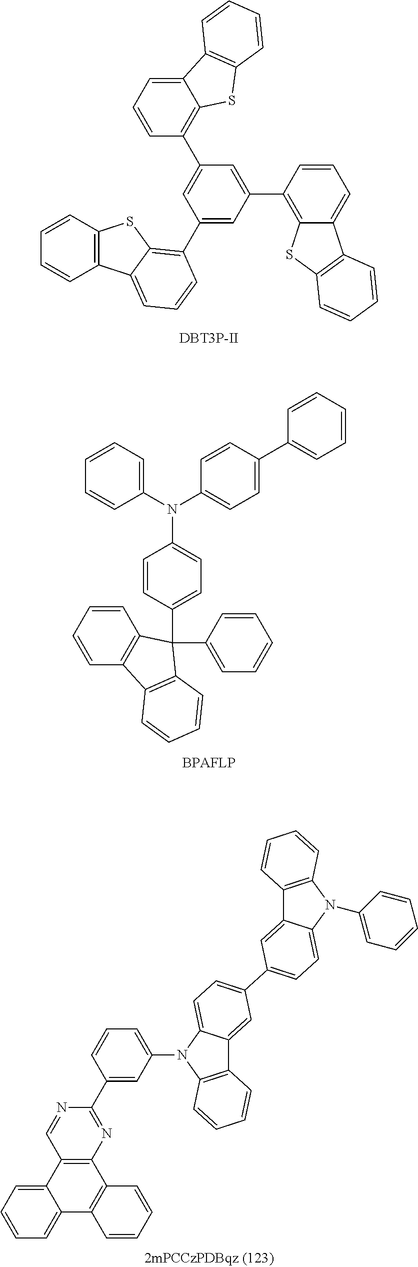

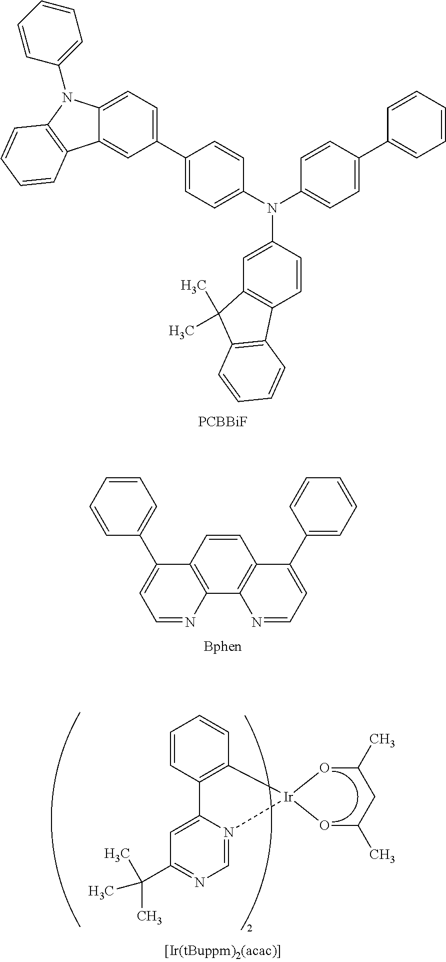

### <<Fabrication of Light-Emitting Element 2>>

Table 3 shows the element structure of Light-emitting Element 2 fabricated in Example 4. As a first electrode of Light-emitting Element 2, indium tin oxide containing silicon oxide (ITO-2) was deposited by a sputtering method. The light-emitting layer and the electron-transport layer of Light-emitting Element 2 were formed using 2mPCCzPDBqz synthesized in Example 3.

TABLE 3

| | | Hole- | Hole- | Light- | | | Electron- | |
|---|---|---|---|---|---|---|---|---|
| | First | injection | transport | emitting | | | injection | Second |
| | electrode | layer | layer | layer | Electron-transport layer | | layer | electrode |
| Light- | ITO-2 | DBT3P-II:MoOx | BPAFLP | * | 2mPCCzPDBqz | Bphen | LiF | Al |
| emitting | (70 nm) | (4:2 60 nm) | (20 nm) | | (20 nm) | (10 nm) | (1 nm) | (200 nm) |
| element 2 | | | | | | | | |

* 2mPCCzPDBqz:PCBBiF:[Ir(tBuppm)₂(acac)] (0.7:0.3:0.05 20 nm\0.8:0.2:0.05 20 nm)

The fabricated light-emitting element 2 was sealed in a glove box under a nitrogen atmosphere so as not to be exposed to the air (specifically, a sealant was applied to surround the element, and at the time of sealing, UV treatment was performed and heat treatment was performed at 80° C. for one hour).

### <<Operation Characteristics of Light-Emitting Element 2>>

Operation characteristics of the fabricated light-emitting element 2 were measured. The measurement was carried out at room temperature (in an atmosphere kept at 25° C.).

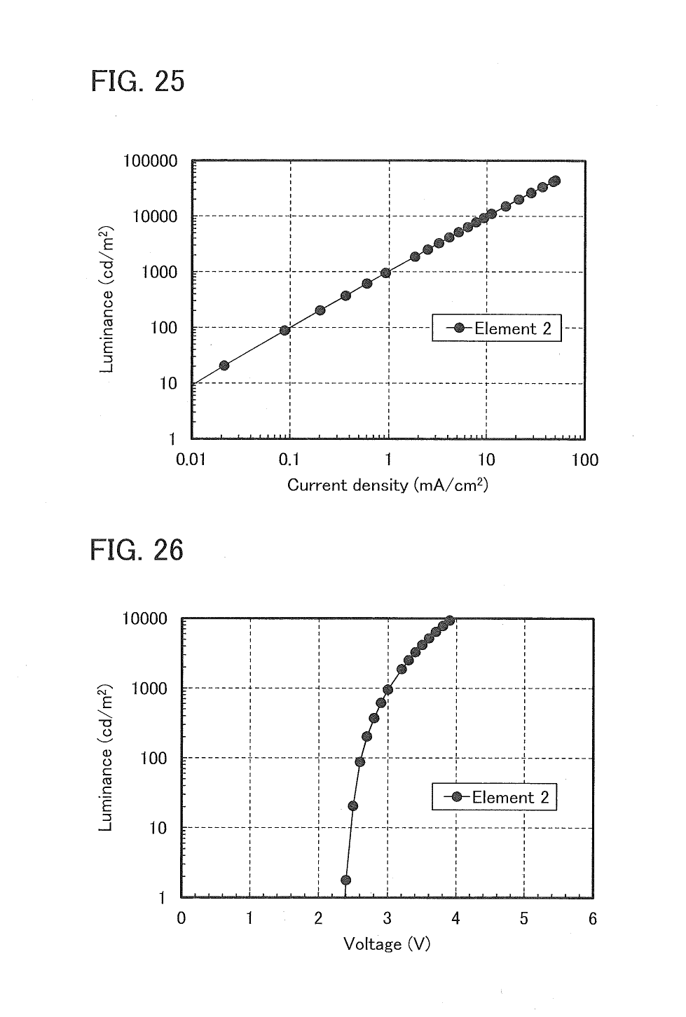

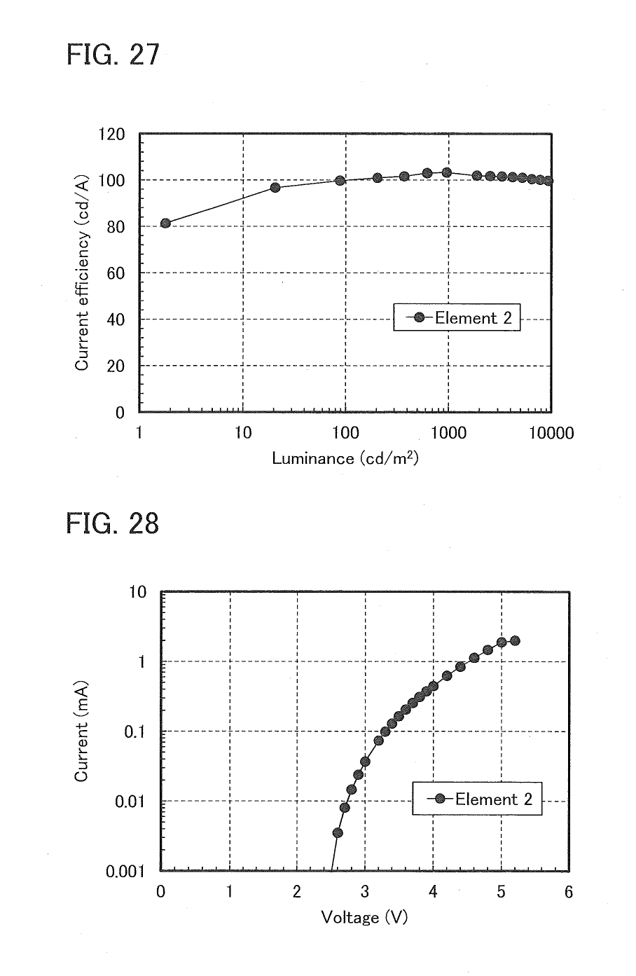

FIG. 25 shows the current density-luminance characteristics of Light-emitting Element 2, FIG. 26 shows the voltage-luminance characteristics of Light-emitting Element 2, FIG. 27 shows the luminance-current efficiency characteristics of Light-emitting Element 2, and FIG. 28 shows the voltage-current characteristics of Light-emitting Element 2.

These results reveal that Light-emitting Element 2 of one embodiment of the present invention has high efficiency. Table 4 shows main values of initial characteristics of Light-emitting Element 2 at a luminance of approximately 1000 cd/m².

TABLE 4

| | | | Current | | | Current | Power | External |
|---|---|---|---|---|---|---|---|---|
| | Voltage | Current | density | Chromaticity | Luminance | efficiency | efficiency | quantum |
| | (V) | (mA) | (mA/cm²) | (x, y) | (cd/m²) | (cd/A) | (lm/W) | efficiency (%) |
| Light- | 3.0 | 0.037 | 0.93 | (0.43, 0.56) | 1000 | 100 | 110 | 28 |
| emitting | | | | | | | | |
| element 2 | | | | | | | | |

The above results show that Light-emitting Element 2 fabricated in Example 4 has high current efficiency and high external quantum efficiency.

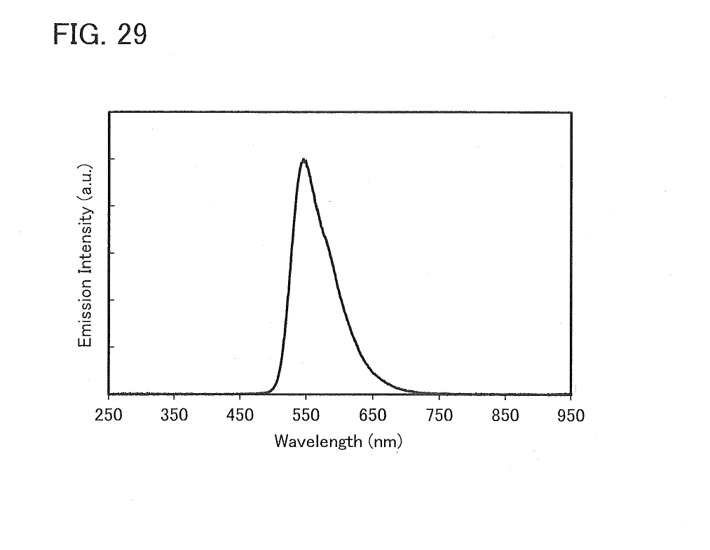

FIG. 29 shows an emission spectrum of Light-emitting Element 2 to which current was applied at a current density of 25 mA/cm². As shown in FIG. 29, the emission spectrum of Light-emitting Element 2 has a peak at around 545 nm, which indicates that the peak is derived from emission of an organometallic complex [Ir(tBuppm)₂(acac)] contained in the light-emitting layer 913. Note that 2mPCCzPDBqz used for the light-emitting layer and the electron-transport layer in Example 4 is the organic compound of one embodiment of the present invention which has a structure in which the 2-position of a dibenzoquinazoline ring is bonded to a skeleton with a hole-transport property via a 1,3-phenylene group and is capable of maintaining the triplet level high; accordingly, a highly efficient element can be obtained. Furthermore, although the difference in band gap and HOMO-LUMO level between a structure in which the 2-position of a dibenzoquinazoline ring is bonded to a skeleton with a hole-transport property via a 1,3-phenylene group and a structure in which the 2-position of a dibenzoquinazoline ring is bonded to a skeleton with a hole-transport property via a 1,4-phenylene group tends to be little, the former structure is suitable for the use as a host material, as compared with the latter structure, because the carrier balance can be easily adjusted and carrier passage can be prevented, so that an element with high efficiency and reliability can be obtained. Thus, the improvement in emission efficiency of Light-emitting Element 2 of Example 4 is attributable to the use of 2mPCCzPDBqz.

This application is based on Japanese Patent Application serial no. 2014-264831 filed with Japan Patent Office on Dec. 26, 2014, the entire contents of which are hereby incorporated by reference.

What is claimed is:

1. A light-emitting element comprising:
an anode;
a cathode; and
an EL layer between the anode and the cathode,
wherein the EL layer comprises a light-emitting layer,
wherein the light-emitting layer comprises a first organic compound and a light-emitting substance, and
wherein the first organic compound contains a dibenzoquinazoline ring and a skeleton with a hole-transport property.

2. The light-emitting element according to claim 1,
wherein the light-emitting layer further comprises a second organic compound, and
wherein the second organic compound has a hole-transport property.

3. A light-emitting device comprising:
the light-emitting element according to claim 1; and
one of a transistor and a substrate.

4. An electronic device comprising:
the light-emitting element according to claim 1; and
one of a connection terminal and an operation key.

5. A lighting device comprising:
the light-emitting element according to claim 1; and
one of a housing, a cover and a support.

6. A light-emitting element comprising:
an anode;
a cathode; and
an EL layer between the anode and the cathode,
wherein the EL layer comprises a light-emitting layer,
wherein the light-emitting layer comprises a first organic compound and a light-emitting substance,
wherein the first organic compound contains a dibenzoquinazoline ring,
wherein a 2-position of the dibenzoquinazoline ring is directly bonded to a first skeleton or is bonded to the first skeleton via one or more arylene groups, and
wherein the first skeleton has a hole-transport property.

7. The light-emitting element according to claim 6, wherein the light-emitting layer further comprises a second organic compound with a hole-transport property.

8. The light-emitting element according to claim 6, wherein the first skeleton is a diarylamino group or a π-electron rich heteroaromatic ring.

9. The light-emitting element according to claim 8, wherein the π-electron rich heteroaromatic ring includes a five-membered heteroaromatic ring.

10. The light-emitting element according to claim 8, wherein the first skeleton is a ring including a dibenzofuran skeleton, a dibenzothiophene skeleton or a carbazole skeleton.

11. The light-emitting element according to claim 6,
wherein the first skeleton contains a first ring and a second ring,
wherein the first ring and the second ring are independently selected from the group consisting of a ring including a dibenzofuran skeleton, a ring including a dibenzothiophene skeleton, and a ring including a carbazole skeleton, and
wherein the first ring and the second ring are bonded to each other.

12. The light-emitting element according to claim 6, wherein the light-emitting substance is a phosphorescent compound.

13. A light-emitting device comprising:
the light-emitting element according to claim 6; and
one of a transistor and a substrate.

14. An electronic device comprising:
the light-emitting element according to claim 6; and
one of a connection terminal and an operation key.

15. A lighting device comprising:
the light-emitting element according to claim 6; and
one of a housing, a cover and a support.

16. An organic compound represented by Formula (G1): 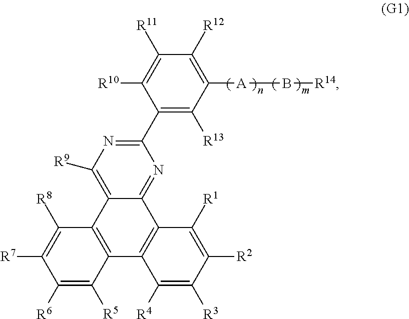
wherein n represents any of 0 to 3,
wherein m represents 1 or 2,
wherein A represents a single bond, or a substituted or unsubstituted arylene group having 6 to 13 carbon atoms,
wherein B represents a ring having a substituted or unsubstituted dibenzofuran skeleton, a ring having a substituted or unsubstituted dibenzothiophene skeleton, or a ring having a substituted or unsubstituted carbazole skeleton, and
wherein each of R¹ to R¹⁴ independently represents any of hydrogen, a substituted or unsubstituted alkyl group having 1 to 6 carbon atoms, a substituted or unsubstituted cycloalkyl group having 5 to 7 carbon atoms, and a substituted or unsubstituted aryl group having 6 to 13 carbon atoms.

17. The organic compound according to claim 16,
wherein B contains one of Formulae (B1) to (B4): 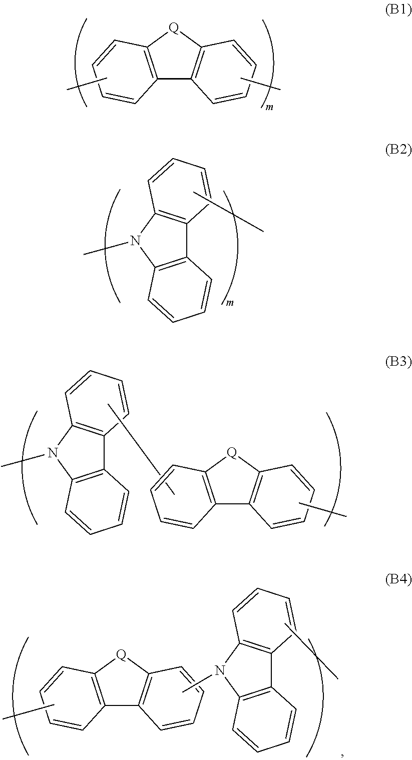
wherein m represents 1 or 2,
wherein Q represents any of O, S and N—R¹⁵, and
wherein R¹⁵ represents hydrogen, or a substituted or unsubstituted phenyl group.

18. The organic compound according to claim 16,
wherein the organic compound is represented by Formula (100) or Formula (123):

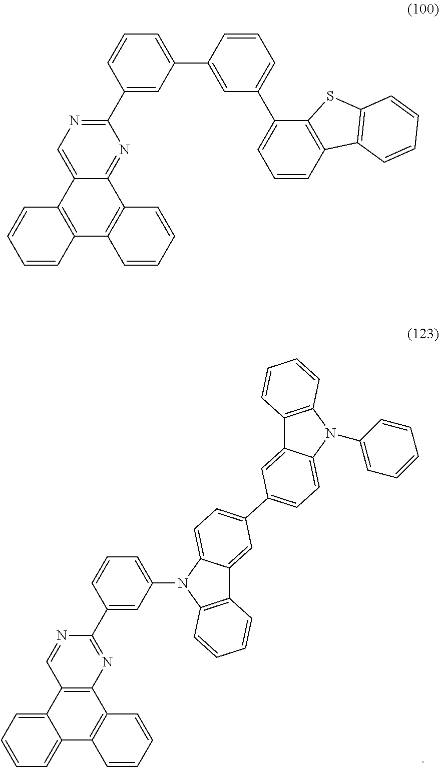 [SMILES: c1cc(-c2cccc(-c3cccc4c3sc3ccccc34)c2)cc(-c2ncc3c4ccccc4c4ccccc4c3n2)c1 ; c1ccc(-n2c3ccccc3c3cc(-c4ccc5c(c4)c4ccccc4n5-c4cccc(-c5ncc6c7ccccc7c7ccccc7c6n5)c4)ccc32)cc1]

19. A light-emitting element comprising the organic compound according to claim 16.

20. A light-emitting device comprising:
the light-emitting element according to claim 19; and
one of a transistor and a substrate.

21. An electronic device comprising:
the light-emitting element according to claim 19; and
one of a connection terminal and an operation key.

22. A lighting device comprising:
the light-emitting element according to claim 19; and
one of a housing, a cover and a support.
